SEQUENCE LISTING (8 sequences)

SEQ ID NO: 1 - protein, 8 aa, Artificial Sequence
  Description of Artificial Sequence Synthetic peptide
GYAFTAYN

SEQ ID NO: 2 - protein, 8 aa, Artificial Sequence
  Description of Artificial Sequence Synthetic peptide
FDPYDGGS

SEQ ID NO: 3 - protein, 7 aa, Artificial Sequence
  Description of Artificial Sequence Synthetic peptide
GWYYFDY

SEQ ID NO: 4 - protein, 6 aa, Artificial Sequence
  Description of Artificial Sequence Synthetic peptide
KSISKY

SEQ ID NO: 5 - protein, 3 aa, Artificial Sequence
  Description of Artificial Sequence Synthetic peptide
SGS

SEQ ID NO: 6 - protein, 8 aa, Artificial Sequence
  Description of Artificial Sequence Synthetic peptide
QQHDESPY

SEQ ID NO: 7 - protein, 446 aa, Artificial Sequence
  Description of Artificial Sequence Synthetic polypeptide
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence
  Description of Artificial Sequence Synthetic polypeptide
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0070988 A1
(43) Pub. Date: Mar. 9, 2023

(54) COMBINATIONS OF EGFR INHIBITORS AND ROR1 INHIBITORS FOR THE TREATMENT OF CANCER

(71) Applicant: ONCTERNAL THERAPEUTICS, INC., San Diego, CA (US)

(72) Inventor: Gunnar KAUFMANN, San Diego, CA (US)

(21) Appl. No.: 17/759,681

(22) PCT Filed: Jan. 29, 2021
(86) PCT No.: PCT/US2021/015749

§ 371 Date: Jul. 28, 2022

Related U.S. Application Data
(60) Provisional application No. 62/968,121, filed on Jan. 30, 2020.
(60) Provisional application No. 63/011,036, filed on Apr. 16, 2020.

(51) Int. Cl. A61K 31/506; A61K 31/501; A61K 31/52; A61K 31/519; A61K 39/395; A61P 35/00

(52) U.S. Cl. CPC A61K 31/506; A61K 31/501; A61K 31/52; A61K 31/519; A61K 39/3955; A61P 35/00; A61K 2039/545

(57) ABSTRACT

Described herein is a method of treating a cancer in an individual comprising administering a tyrosine kinase-like orphan receptor 1 (ROR1) antagonist and an epidermal growth factor receptor (EGFR) inhibitor. In some embodiments, the ROR1 antagonist is cirmtuzumab. In some embodiments, the EGFR inhibitor is osimertinib. In some embodiments, the cancer is a lung cancer such as a non-small cell lung cancer.

### CROSS-REFERENCE TO RELATED APPLICATIONS

This application claims the benefit of U.S. Provisional Application Ser. No. 62/968,121, filed on Jan. 30, 2020; and U.S. Provisional Application Ser. No. 63/011,036, filed on Apr. 16, 2020, both of which are incorporated by reference herein in their entirety.

### SUMMARY

The compositions and methods provided herein are, inter alia, useful for the treatment of cancer. For example, provided herein are surprisingly effective methods for using the combination of an ROR1 antagonist, such as an anti-ROR1 antibody, with EGFR inhibitors to treat cancers, including non-small cell lung cancer (NSCLC), breast cancer, glioma, head and neck cancer, pancreatic cancer, hepatocellular carcinoma, and biliary tract cancer. Also provided herein is the use of a third-generation EGFR inhibitor in combination with an anti-ROR1 antibody to treat a cancer. The methods, uses and compositions described herein may additionally be useful for use in treating cancers and or tumors that have developed resistance to first, second- and/or third-generation EGFR inhibitors.

In an aspect is provided a method of treating cancer in a subject in need thereof, the method including administering to the subject a therapeutically effective amount of an EGFR inhibitor and a tyrosine kinase-like orphan receptor 1 (ROR1) antagonist.

In an aspect is provided a pharmaceutical composition including an EGFR inhibitor, a ROR1 antagonist and a pharmaceutically acceptable excipient.

In an aspect is provided a pharmaceutical composition including an EGFR inhibitor, an anti-ROR1 antibody and a pharmaceutically acceptable excipient, wherein EGFR inhibitor and the anti-ROR1 antibody are present in a combined synergistic amount, wherein the combined synergistic amount is effective to treat cancer in a subject in need thereof.

In an aspect, there is provided a method of treating cancer in a subject in need thereof. The method includes administering to the subject a therapeutically effective amount of an EGFR inhibitor and an anti-ROR1 antibody.

In another aspect, there is provided a pharmaceutical composition including an EGFR inhibitor, an anti-ROR1 antibody, and a pharmaceutically acceptable excipient.

In one aspect the ROR1 antibody comprises cirmtuzumab or an antigen binding fragment thereof and the EGFR inhibitor comprises osimertinib.

In certain embodiments, these methods result in reduced toxicity compared to administration of ROR1 antagonists, such as cirmtuzumab, when administered as a monotherapy.

In an aspect is provided a method of treating cancer in a subject in need thereof, the method including administering to the subject a therapeutically effective amount of an EGFR inhibitor and a tyrosine kinase-like orphan receptor 1 (ROR1) antagonist. In certain embodiments, said EGFR inhibitor is a small molecule. In certain embodiments, said EGFR inhibitor is a third-generation EGFR inhibitor. In certain embodiments, said third-generation EGFR inhibitor is osimertinib, AC0010, lapatinib, mavelertinib, naquotinib, nazartinib, olmutinib, or rociletinib. In certain embodiments, said EGFR inhibitor is osimertinib. In certain embodiments, said ROR1 antagonist is an antibody or a small molecule. In certain embodiments, said antibody comprises a Fab, F(ab′)2, Fv, or an scFv. In certain embodiments, said ROR1 antagonist is an anti-ROR1 antibody. In certain embodiments, antibody comprises a humanized heavy chain variable region and a humanized light chain variable region, wherein said humanized heavy chain variable region comprises the sequences set forth in SEQ ID NO:1, SEQ ID NO:2, and SEQ ID NO:3; and wherein said humanized light chain variable region comprises the sequences set forth in SEQ ID NO:4, SEQ ID NO:5, and SEQ ID NO:6. In certain embodiments, said antibody comprises a heavy chain variable region and a light chain variable region, wherein said heavy chain variable region comprises an amino acid sequence at least about 85%, 90%, 95%, 97%, 98%, 99%, or 100% identical to that set forth in SEQ ID NO: 7; and wherein said light chain variable region comprises an amino acid sequence at least about 85%, 90%, 95%, 97%, 98%, 99%, or 100% identical to that set forth in SEQ ID NO: 8. In certain embodiments, said antibody is cirmtuzumab. In certain embodiments, said individual is afflicted with a cancer that comprises a mutated EGFR gene. In certain embodiments, the mutated EGFR gene comprises a mutation resulting in a T790M mutation or an L858R mutation in the EGFR protein or an exon-20 insertion in the EGFR gene. In certain embodiments, said EGFR inhibitor and said ROR1 antagonist are administered in a combined synergistic amount. In certain embodiments, said EGFR inhibitor and said ROR1 antagonist are administered substantially simultaneously. In certain embodiments, said EGFR inhibitor and said ROR1 antagonist are administered separately. In certain embodiments, said EGFR inhibitor and said ROR1 antagonist are administered in separate compositions. In certain embodiments, said ROR1 antagonist is administered at a first time point and said EGFR inhibitor is administered at a second time point, wherein said first time point precedes said second time point. In certain embodiments, said EGFR inhibitor and said ROR1 antagonist are admixed prior to administration. In certain embodiments, said EGFR inhibitor is administered at an amount from about 20 mg to about 100 mg daily. In certain embodiments, said EGFR inhibitor is administered at an amount of about 80 mg daily. In certain embodiments, said EGFR inhibitor is administered at an amount of less than about 80 mg daily. In certain embodiments, said EGFR inhibitor is administered intravenously. In certain embodiments, said EGFR inhibitor is administered orally. In certain embodiments, said EGFR inhibitor is administered daily. In certain embodiments, said ROR1 antagonist is administered intravenously. In certain embodiments, said ROR1 antagonist is administered once every two-weeks. In certain embodiments, said ROR1 antagonist is administered once every three-weeks. In certain embodiments, said ROR1 antagonist is administered once every four-weeks. In certain embodiments, said ROR1 antagonist is administered at a dosage from about 200 milligrams to about 800 milligrams. In certain embodiments, said ROR1 antagonist is administered at a dosage from about 300 milligrams to about 600 milligrams. In certain embodiments, said ROR1 antagonist is administered at a dosage of about 300 milligrams. In certain embodiments, said ROR1 antagonist is administered at a dosage of about 600 milligrams. In certain embodiments, said subject is a mammal. In certain embodiments, said subject is a human. In certain embodiments, said cancer is lymphoma, leukemia, myeloma, Acute myelogenous leukemia (AML), T-ALL, renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, or adrenal cancer. In certain embodiments, the cancer is a non-small cell lung cancer. In certain embodiments, the non-small cell lung cancer comprises a mutation resulting in a T790M mutation or an L858R mutation in the EGFR protein or an exon-20 insertion in the EGFR gene. In certain embodiments, the cancer is a breast cancer. In certain embodiments, said cancer is chronic lymphocytic leukemia (CLL), small lymphocytic lymphoma (SLL), marginal cell B-Cell lymphoma (MZL), diffuse large B-cell lymphoma (DLBCL), Burkitt's Lymphoma, B cell acute lymphocytic leukemia, or B cell leukemia.

Also described is a pharmaceutical composition comprising an EGFR inhibitor, an ROR1 antagonist, and a pharmaceutically acceptable excipient.

The methods and uses described herein may be for use in treating a cancer wherein said cancer is renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, biliary cancer, or adrenal cancer. In certain embodiments, the cancer is colon adenocarcinoma. In certain embodiments, the cancer is cutaneous melanoma. In certain embodiments, the cancer is glioblastoma multiforme. In certain embodiments, the lung cancer is lung adenocarcinoma. In certain embodiments, the cancer is a non-small cell lung cancer. In certain embodiments, the non-small cell lung cancer comprises a mutation. In certain embodiments, the cancer is a breast cancer. In certain embodiments, the cancer has exhibited resistance to a third-generation EGFR inhibitor as a monotherapy.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A shows inhibition of tumor growth in mice inoculated with LU0858 tumors and treated as described.

FIG. 1B shows illustrates shows % inhibition of tumor growth in mice inoculated with LU0858 tumors and treated as described.

FIG. 1C shows body weights in mice inoculated with LU0858 tumors and treated as described.

FIG. 2 shows a dose response curve for NCI-H1975 cells treated in vitro with various amounts of EGFR inhibitors or of an ROR1 antagonist.

FIG. 3A shows tumor growth in mice inoculated with NCI-H1975 and treated with vehicle or erlotinib.

FIG. 3B shows tumor growth in mice inoculated with NCI-H1975 and treated with vehicle or gefitinib.

FIG. 3C shows tumor growth in mice inoculated with NCI-H1975 and treated with vehicle or afatinib.

FIG. 3D shows tumor growth or inhibition of tumor growth in mice inoculated with NCI-H1975 and treated with vehicle or osimertinib (AZD9291).

FIG. 4 shows gene expression of ROR1 and WNT5A in NCI-H1975 cells.

FIG. 5A shows tumor growth in mice inoculated with NCI-H1975 and treated with vehicle, cirmtuzumab (UC961), erlotinib, gefitinib, afatinib, erlotinib and cirmtuzumab, gefitinib and cirmtuzumab, or afatinib and cirmtuzumab.

FIG. 5B shows % inhibition of tumor growth in mice inoculated with NCI-H1975 and treated with cirmtuzumab (UC961), erlotinib, gefitinib, afatinib, erlotinib and cirmtuzumab, gefitinib and cirmtuzumab, or afatinib and cirmtuzumab.

FIG. 6A shows inhibition of tumor growth in mice inoculated with LU3075 tumors and treated as described.

FIG. 6B shows body weights in mice inoculated with LU3075 tumors and treated as described.

### DETAILED DESCRIPTION

Described herein in one aspect is a method of treating cancer in a subject in need thereof, said method comprising administering to said subject a therapeutically effective amount of an epidermal growth factor receptor (EGFR) inhibitor and a tyrosine kinase-like orphan receptor 1 (ROR1) antagonist. In certain embodiments, said EGFR inhibitor is a small molecule. In certain embodiments, said EGFR inhibitor is a third-generation EGFR inhibitor. In certain embodiments, said EGFR inhibitor is erlotinib, gefitinib, afatinib, or osimertinib. In certain embodiments, said EGFR inhibitor is osimertinib. In certain embodiments, said ROR1 antagonist is an antibody or a small molecule. In certain embodiments, said ROR1 antagonist is an anti-ROR1 antibody. In certain embodiments, said antibody comprises a humanized heavy chain variable region and a humanized light chain variable region, wherein said humanized heavy chain variable region comprises the sequences set forth in SEQ ID NO:1, SEQ ID NO:2, and SEQ ID NO:3; and wherein said humanized light chain variable region comprises the sequences set forth in SEQ ID NO:4, SEQ ID NO:5, and SEQ ID NO:6. In certain embodiments, said antibody is cirmtuzumab. In certain embodiments, said individual is afflicted with a cancer that comprises a mutated EGFR gene. In certain embodiments, the mutated EGFR gene comprises a mutation resulting in a T790M mutation or an L858R mutation in the EGFR protein or an exon-20 insertion in the EGFR gene. In certain embodiments, said EGFR inhibitor and said ROR1 antagonist are administered in a combined synergistic amount. In certain embodiments, said EGFR inhibitor and said ROR1 antagonist are administered simultaneously or sequentially. In certain embodiments, said ROR1 antagonist is administered at a first time point and said EGFR inhibitor is administered at a second time point, wherein said first time point precedes said second time point. In certain embodiments, said EGFR inhibitor and said ROR1 antagonist are admixed prior to administration. In certain embodiments, said EGFR inhibitor is administered at an amount of about from about 20 mg to about 100 mg daily. In certain embodiments, said EGFR inhibitor is administered at an amount of about 80 mg daily. In certain embodiments, said EGFR inhibitor is administered at an amount of less than about 80 mg daily. In certain embodiments, said EGFR inhibitor is administered intravenously. In certain embodiments, said ROR1 antagonist is administered intravenously. In certain embodiments, said subject is a mammal. In certain embodiments, said antibody comprises a heavy chain variable region and a light chain variable region, wherein said heavy chain variable region comprises an amino acid sequence at least about 85%, 90%, 95%, 97%, 98%, 99%, or 100 identical to that set forth in SEQ ID NO: 7; and wherein said light chain variable region comprises an amino acid sequence at least about 85%, 90%, 95%, 97%, 98%, 99%, or 100 identical to that set forth in SEQ ID NO: 8. In certain embodiments, said antibody comprises a Fab, F(ab′)₂, Fv, or an scFv. In certain embodiments, the cancer is a non-small cell lung cancer. In certain embodiments, the non-small cell lung cancer comprises a mutation resulting in a T790M mutation or an L858R mutation in the EGFR protein or an exon-20 insertion in the EGFR gene. In certain embodiments the cancer is renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, biliary cancer, or adrenal cancer. In certain embodiments, the cancer is colon adenocarcinoma. In certain embodiments, the cancer is cutaneous melanoma. In certain embodiments, the cancer is glioblastoma multiforme. In certain embodiments, the lung cancer is lung adenocarcinoma. In certain embodiments, the cancer is a non-small cell lung cancer. In certain embodiments, the non-small cell lung cancer comprises a mutation. In certain embodiments, the cancer is a breast cancer. In certain embodiments, the cancer has exhibited resistance to a third-generation EGFR inhibitor as a monotherapy.

Some embodiments relate to administration of an EGFR inhibitor such as a third-generation EGFR inhibitor and a ROR1 antagonist. In some cases, the combined treatment is advantageous and results in greater effectiveness in the treatment. In some cases, an advantage of the combined treatment is decreased side-effects or adverse effects associated with treatment of either agent alone because the combined treatment may include a dose of the EGFR inhibitor or the ROR1 antagonist that is below a manufacturer-recommended or FDA-approved dose, and the reduced dose may be associated with reduced side effects. For example, the combined treatment of mice with cirmtuzumab and osimertinib was effective for reducing tumor growth without observable adverse effects such as toxicity or weight loss.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

“Treatment” or “treating” refers to administration of or administering medical care to a subject. A treatment generally refers to an intervention designed to ameliorate one or more symptoms of a disorder. For example, treating may include administering an EGFR inhibitor and a ROR1 antagonist to a cancer patient in an attempt to ameliorate one or more symptoms of the cancer. Treating may include prevention, inhibition, or reversion of a disorder as a result of the medical care. For example, treating cancer can include preventing cancer recurrence, inhibiting cancer growth or a cancer symptom, or reversing the development of the cancer or a symptom thereof, in response to administration of the medical care. Additional examples may include inhibiting cancer cell growth, inhibiting cancer cell division, increasing cancer cell death, inhibiting tumor growth, decreasing tumor volume or slowing an increase in tumor volume, inhibiting tumor size or slowing an increase in tumor size, inhibitory effects on tumor diameter, inhibitory effects on tumor width, inhibitory effects on tumor length, inhibitory effects on tumor burden, decreasing metastasis, decreasing a number of cancer cells, improving survival, or treating another cancer symptom. Treatment with reference to cancer can comprise inducing a complete response, a partial response, or stable disease.

The terms “cirmtuzumab”, “UC-961”, and “99961.1” are used interchangeably, and refer to a humanized monoclonal antibody capable of binding the extracellular domain of the human receptor tyrosine kinase-like orphan receptor 1 (ROR1). Some embodiments include, cirmtuzumab or any one of the antibodies or fragments thereof disclosed in U.S. Pat. Nos. 9,758,951 and 10,344,096, which is incorporated by reference herein in its entirety and for all purposes. “ROR1” and “ROR1” are used interchangeably.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

Among the provided antibodies are monoclonal antibodies, polyclonal antibodies, multispecific antibodies (for example, bispecific antibodies and polyreactive antibodies), and antibody fragments. The antibodies may include antibody-conjugates and molecules comprising the antibodies, such as chimeric molecules. Thus, an antibody may include, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody may include a composition of substantially homogeneous antibodies. In some embodiments, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region. A polyclonal antibody may include a preparation that includes different antibodies of varying sequences that generally are directed against two or more different determinants (epitopes).

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR—H1, FR—H2, FR—H3, and FR—H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, the polypeptide or protein includes an antibody. In some embodiments, the polypeptide or protein includes a polypeptide or protein other than an antibody. In some embodiments, the polypeptide or protein includes a antagonist or inhibitor of an epidermal growth factor (EGFR) or tyrosine kinase-like orphan receptor 1 (ROR1).

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, amino acid sequence variants of the antibodies or polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the disclosure and evaluating one or more biological activities of the polypeptide as described herein and/or using any of a number of known techniques. For example, it may be desirable to improve the binding affinity and/or other biological properties of the antibody or polypeptide amino acid sequence. Variants of an antibody or polypeptide may be prepared by introducing appropriate modifications into the nucleotide sequence encoding the antibody or polypeptide, or by peptide synthesis. Such modifications include, for example, deletions from, and/or insertions into and/or substitutions of residues within the amino acid sequences of the antibody or polypeptide. Any combination of deletion, insertion, and substitution can be made to arrive at the final construct, provided that the final construct possesses the desired characteristics, e.g., antigen-binding.

In some embodiments, antibody or polypeptide variants having one or more amino acid substitutions are provided. Sites of interest for mutagenesis by substitution include the CDRs and FRs. Amino acid substitutions may be introduced into an antibody or polypeptide of interest and the products screened for a desired activity, e.g., retained/improved antigen binding, decreased immunogenicity, or improved ADCC or CDC.

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, the antibodies or polypeptides are altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn₂₉₇ of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn297 (See e.g., WO 08/077546). Asn₂₉₇ refers to the asparagine residue located at about position 297 in the Fc region (EU numbering of Fc region residues; See e.g., Edelman et al. Proc Natl Acad Sci USA. 1969 May; 63(1):78-85). However, Asn₂₉₇ may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87: 614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87: 614 (2004); Kanda, Y. et al., Biotechnol. Bioeng., 94(4):680-688 (2006)). Other antibody glycosylation variants are also included (See e.g., U.S. Pat. No. 6,602,684).

In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or less (e.g., 10⁻⁸ M or less, e.g., from 10⁻⁸ M to 10⁻¹³ M, e.g., from 10⁻⁹ M to 10⁻¹³ M) for the antibody target. The antibody target can be ROR1. K_{D} can be measured by any suitable assay. In certain embodiments, KD can be measured using surface plasmon resonance assays (e.g., using a BIACORE®-2000 or a BIACORE®-3000 or Octet).

In some embodiments, one or more amino acid modifications may be introduced into the Fc region of an antibody provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the antibodies or polypeptides of this disclosure are variants that possess some but not all effector functions, which make it a desirable candidate for applications in which the half-life of the antibody or polypeptide in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., U.S. 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody or polypeptide provided herein may be further modified to contain additional nonproteinaceous moieties that are known and available. The moieties suitable for derivatization of the antibody or polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody or polypeptide may vary, and if two or more polymers are attached, they can be the same or different molecules.

The antibodies and polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antibodies or polypeptides described herein can be used to infect, transfect, transform, or otherwise render a suitable cell transgenic for the nucleic acid, thus enabling the production of antibodies or polypeptides for commercial or therapeutic uses. Standard cell lines and methods for the production of antibodies or polypeptides from a large scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies or polypeptides is a Chines Hamster Ovary cell (CHO) cell, an NS0 murine myeloma cell, or a PER.C6® cell. In certain embodiments, the nucleic acid encoding the antibody or polypeptide is integrated into a genomic locus of a cell useful for producing the antibodies or polypeptides. In certain embodiments, described herein is a method of making an antibody or polypeptide comprising culturing a cell comprising a nucleic acid encoding the antibody or polypeptide under conditions in vitro sufficient to allow production and secretion of said antibody or polypeptide.

### EGFR Inhibitors

Treatment with epidermal growth factor receptor (EGFR) inhibitors may be used in combination with ROR1 antagonists for combatting cancer. Several EGFR inhibitors are available, and multiple-generations of EGFR inhibitors have been developed to combat cancers that are resistant to earlier forms of EGFR inhibitors. Third-generation, or newer, EGFR inhibitors such as osimertinib may be particularly efficacious in combined treatments with an ROR1 antagonist generally, or cirmtuzumab specifically.

In certain embodiments, disclosed herein, are EGFR inhibitors or antagonists. Some embodiments relate to an EGFR antagonist. Some embodiments relate to an EGFR inhibitor. In some embodiments, the EGFR inhibitor is or includes a polypeptide. In some embodiments, the EGFR inhibitor is or includes an antibody. In some embodiments, the EGFR inhibitor is or includes a fusion protein. In some embodiments, the EGFR inhibitor is or includes a small molecule EGFR inhibitor. In some embodiments, the EGFR inhibitor is or includes an oligonucleotide EGFR inhibitor such as an antisense oligonucleotide or small interfering RNA (siRNA). Some embodiments include a salt of a known EGFR inhibitor.

Examples of EGFR inhibitors include osimertinib, AC0010, afatinib, cetuximab, dacomitinib, EAI045, erlotinib, gefitinib, lapatinib, mavelertinib, naquotinib, nazartinib, necitumumab, neratinib, panitumumab, olmutinib, rociletinib, and vandetanib. In some embodiments, the EGFR inhibitor comprises osimertinib. In some embodiments, the EGFR inhibitor comprises afatinib. In some embodiments, the EGFR inhibitor comprises cetuximab. In some embodiments, the EGFR inhibitor comprises dacomitinib. In some embodiments, the EGFR inhibitor comprises erlotinib. In some embodiments, the EGFR inhibitor comprises gefitinib. In some embodiments, the EGFR inhibitor comprises lapatinib. In some embodiments, the EGFR inhibitor comprises necitumumab. In some embodiments, the EGFR inhibitor comprises neratinib. In some embodiments, the EGFR inhibitor comprises panitumumab. In some embodiments, the EGFR inhibitor comprises rociletinib. In some embodiments, the EGFR inhibitor comprises vandetanib. In some embodiments, the EGFR inhibitor comprises AC0010. In some embodiments, the EGFR inhibitor comprises mavelertinib. In some embodiments, the EGFR inhibitor comprises naquotinib. In some embodiments, the EGFR inhibitor comprises nazartinib. In some embodiments, the EGFR inhibitor comprises olmutinib. In some embodiments, the EGFR inhibitor comprises EAI045.

In some embodiments, the EGFR inhibitor comprises a tyrosine kinase inhibitor. Examples of tyrosine kinase inhibitors include osimertinib, erlotinib and gefitinib. In some embodiments, the tyrosine kinase inhibitor binds to a tyrosine kinase domain in the EGFR and/or stops or decreases activity of the EGFR. In some embodiments, the EGFR inhibitor comprises an antibody. In some embodiments, the antibody is a monoclonal antibody. Examples of some such monoclonal antibodies include cetuximab, necitumumab, and panitumumab. In some embodiments, the antibody is a polyclonal antibody. In some embodiments, the EGFR inhibitor (e.g. monoclonal anti-EGFR antibody) binds to an extracellular component of EGFR, prevents epidermal growth factor from binding to the EGFR, and/or prevents activation of EGFR signaling. In some embodiments, the EGFR inhibitor comprises an allosteric EGFR inhibitor. In some embodiments, the EGFR inhibitor comprises a dual tyrosine kinase inhibitor. The dual tyrosine kinase inhibitor may inhibit HER2 signaling in addition to EGFR.

The EGFR inhibitor may include a first-generation EGFR inhibitor. In some embodiments, the EGFR inhibitor includes a first-generation EGFR inhibitor such as erlotinib or gefitinib.

The EGFR inhibitor may include a second-generation EGFR inhibitor. Second-generation EGFR inhibitors were developed for the advantage of combatting resistance to first-generation EGFR inhibitors. In some embodiments, the EGFR inhibitor includes a second-generation EGFR inhibitor such as afatinib, dacomitinib, neratinib, or vandetanib. In some embodiments, the second-generation EGFR inhibitor is a covalent EGFR inhibitor. The covalent EGFR inhibitor may be a reversible covalent inhibitor, or it may be an irreversible covalent EGFR inhibitor.

The EGFR inhibitor may include a third-generation EGFR inhibitor. Third-generation EGFR inhibitors were developed for the advantage of combatting resistance to second-generation EGFR inhibitors. Third-generation EGFR inhibitors may be designed to overcome EGFR T790M mutations that may lead to resistance to other EGFR inhibitors such as first and second-generation EGFR inhibitors. In some embodiments, the EGFR inhibitor includes a third-generation EGFR inhibitor such as AC0010, lapatinib, mavelertinib, naquotinib, nazartinib, olmutinib, osimertinib or rociletinib. In some embodiments, the third-generation EGFR inhibitor comprises osimertinib. In some embodiments, the third-generation EGFR inhibitor consists of osimertinib. In some embodiments, the third-generation EGFR inhibitor is a covalent EGFR inhibitor. The covalent EGFR inhibitor may be a reversible covalent inhibitor, or it may be an irreversible covalent EGFR inhibitor.

The embodiments described are not intended to be limiting. For example, an embodiment encompassing a third-generation EGFR inhibitor is not intended to exclude fourth or later-generations that include features of third-generation EGFR inhibitors. For example, the EGFR inhibitor may include an allosteric EGFR C797S inhibitor. In some embodiments, the EGFR inhibitor is or includes a fourth-generation EGFR inhibitor. An example of a fourth-generation EGFR inhibitor is EAI045.

In some embodiments, the EGFR inhibitor inhibits the activity of EGFR. In some embodiments, the EGFR inhibitor inhibits the expression of an EGFR protein. In some embodiments, the EGFR inhibitor increases the degradation of an EGFR protein. In some embodiments, the EGFR inhibitor inhibits the expression of an EGFR transcript.

In some embodiments, the EGFR inhibitor has an inhibitory effect such as inhibiting EGFR activity, inhibiting the expression of an EGFR protein, increasing the degradation of an EGFR protein, or inhibiting the expression of an EGFR transcript. In some embodiments, the inhibitory effect comprises an inhibitory effect on cell growth. In some embodiments, the inhibitory effect comprises an inhibitory effect on cell division. In some embodiments, the inhibitory effect comprises an increase in cell death. In some embodiments, the cell death comprises apoptosis. In some embodiments, the inhibitory effect comprises an inhibitory effect on tumor growth. In some embodiments, the inhibitory effect comprises an inhibitory effect on tumor volume. In some embodiments, the inhibitory effect comprises an inhibitory effect on tumor size. In some embodiments, the inhibitory effect comprises an inhibitory effect on tumor diameter. In some embodiments, the inhibitory effect comprises an inhibitory effect on tumor width. In some embodiments, the inhibitory effect comprises an inhibitory effect on tumor length. In some embodiments, the inhibitory effect comprises an inhibitory effect on tumor burden. In some embodiments, the inhibitory effect comprises an inhibitory effect on metastasis. In some embodiments, the inhibitory effect comprises an inhibitory effect on an amount of cancer cells.

In some embodiments, the inhibitory effect is 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, or a range of percentages defined by any two of the aforementioned percentages. In some embodiments, the inhibitory effect is about 1%, about 2.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98% about 99%, or about 100%, or a range of percentages defined about by any two of the aforementioned percentages. In some embodiments, the inhibitory effect is less than 1%, less than 2.5%, less than 5%, less than less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 96%, less than 97%, less than 98%, less than 99%, or less than 100%. In some embodiments, the inhibitory effect is greater than 1%, greater than 2.5%, greater than 5%, greater than greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 96%, greater than 97%, greater than 98%, greater than 99%, or greater than 100%.

In some embodiments, the inhibitory effect is relative to a control. In some embodiments, the control is a subject with a cancer or tumor who is untreated. In some embodiments, the control is a subject with a cancer or tumor who is treated with a vehicle. In some embodiments, the control is a subject with a cancer or tumor who is treated with a compound other than an EGFR inhibitor. In some embodiments, the control is a subject without cancer. In some embodiments, the control is a subject without a tumor. In some embodiments, the control is a population or group of such subjects.

In some embodiments, the EGFR inhibitor is formulated as a pharmaceutical composition. In some embodiments, the EGFR inhibitor is formulated in combination with a ROR1 antagonist or inhibitor. In some embodiments, the EGFR inhibitor is formulated for treatment of a subject with a cancer (e.g. a lung cancer such as non-small cell lung cancer). In some embodiments, the EGFR inhibitor is formulated for administration to a subject with cancer in combination with a ROR1 antagonist or inhibitor.

### ROR1 Antagonists

Treatment with tyrosine kinase-like orphan receptor 1 (ROR1) antagonists may be used in combination with EGFR inhibitors for combatting cancer. Antibodies such as cirmtuzumab may be particularly efficacious in combined treatments.

In certain embodiments, disclosed herein, are ROR1 inhibitors or antagonists. Some embodiments relate to a ROR1 inhibitor. Some embodiments relate to a ROR1 antagonist. In some embodiments, the ROR1 antagonist is or includes a polypeptide. In some embodiments, the ROR1 antagonist is or includes an antibody. In some embodiments, the ROR1 antagonist is or includes a fusion protein. In some embodiments, the ROR1 antagonist is or includes a small molecule ROR1 antagonist. In some embodiments, the ROR1 inhibitor is or includes an oligonucleotide ROR1 inhibitor such as an antisense oligonucleotide or siRNA. Some embodiments include a salt of a known ROR1 inhibitor or antagonist.

Examples of ROR1 antagonists or inhibitors include cirmtuzumab, ARI-1, KAN0439834, and strictinin. In some embodiments, the ROR1 antagonist comprises cirmtuzumab. In some embodiments, the ROR1 inhibitor comprises ARI-1. In some embodiments, the ROR1 inhibitor comprises KAN0439834. In some embodiments, the ROR1 inhibitor comprises strictinin.

In some embodiments, the ROR1 antagonists comprise a tyrosine kinase inhibitor. Examples of some such tyrosine kinase inhibitors include KAN0439834. In some embodiments, the tyrosine kinase inhibitor binds to a tyrosine kinase domain in the ROR1 and/or stops or decreases activity of the ROR1. In some embodiments, the ROR1 antagonist comprises an antibody. In some embodiments, the antibody is a monoclonal antibody. Examples of some such monoclonal antibodies include cirmtuzumab. In some embodiments, the antibody is a polyclonal antibody. In some embodiments, the ROR1 inhibitor (e.g. monoclonal anti-ROR1 antibody) binds to an extracellular component of ROR1, prevents WNT5A from binding to the ROR1, and/or prevents or reduces activation of ROR1 signaling.

In some embodiments, the antibody comprises a humanized antibody. In some embodiments, the monoclonal antibody comprises a humanized antibody. In some embodiments, the monoclonal antibody comprises a heavy chain variable region. In some embodiments, the heavy chain variable region comprises a sequence set forth in SEQ ID NO: 1. In some embodiments, the heavy chain variable region comprises a sequence set forth in SEQ ID NO: 2. In some embodiments, the heavy chain variable region comprises a sequence set forth in SEQ ID NO: 3. In some embodiments, the heavy chain variable region comprises a sequence set forth in SEQ ID NO: 7. In some embodiments, the heavy chain variable region comprises an amino acid sequence at least about 85%, 90%, 95%, 97%, 98%, 99%, or 100 identical to that set forth in SEQ ID NO: 7. In some embodiments, the heavy chain variable region comprises a sequence set forth in SEQ ID NO: 1, SEQ ID NO: 2, SEQ ID NO: 3, or SEQ ID NO: 7. In some embodiments, the monoclonal antibody comprises a humanized heavy chain variable region. In some embodiments, the humanized heavy chain variable region comprises a sequence set forth in SEQ ID NO: 1, SEQ ID NO: 2, and/or SEQ ID NO: 3.

In some embodiments, the monoclonal antibody comprises a light chain variable region. In some embodiments, the light chain variable region comprises a sequence set forth in SEQ ID NO: 4. In some embodiments, the light chain variable region comprises a sequence set forth in SEQ ID NO: 5. In some embodiments, the light chain variable region comprises a sequence set forth in SEQ ID NO: 6. In some embodiments, the light chain variable region comprises a sequence set forth in SEQ ID NO: 8. In some embodiments, the light chain variable region comprises an amino acid sequence at least about 85%, 90%, 95%, 97%, 98%, 99%, or 100 identical to that set forth in SEQ ID NO: 8. In some embodiments, the light chain variable region comprises a sequence set forth in SEQ ID NO: 4, SEQ ID NO: 5, SEQ ID NO: 6, or SEQ ID NO: 8. In some embodiments, the monoclonal antibody comprises a humanized light chain variable region. In some embodiments, the humanized light chain variable region comprises a sequence set forth in SEQ ID NO: 4, SEQ ID NO: 5, and/or SEQ ID NO: 6.

Other antibodies and antibody fragments that bind and inhibit ROR1 function can be found in for example in U.S. Pat. Nos. 9,933,434; 9,938,350; 9,266,952; 9,758,586; 9,316,646; or U.S. Pat. No. 9,228,023.

In some embodiments, the ROR1 inhibitor or antagonist inhibits the activity of ROR1. In some embodiments, the ROR1 inhibitor inhibits the expression of a ROR1 protein. In some embodiments, the ROR1 inhibitor increases the degradation of a ROR1 protein. In some embodiments, the ROR1 inhibitor inhibits the expression of a ROR1 transcript.

In some embodiments, the ROR1 inhibitor or antagonist has an inhibitory effect such as inhibiting ROR1 activity, inhibiting the expression of an ROR1 protein, increasing the degradation of an ROR1 protein, or inhibiting the expression of an ROR1 transcript. In some embodiments, the inhibitory effect comprises an inhibitory effect on cell growth. In some embodiments, the inhibitory effect comprises an inhibitory effect on cell division. In some embodiments, the inhibitory effect comprises an increase in cell death. In some embodiments, the cell death comprises apoptosis. In some embodiments, the inhibitory effect comprises an inhibitory effect on tumor growth. In some embodiments, the inhibitory effect comprises an inhibitory effect on tumor volume. In some embodiments, the inhibitory effect comprises an inhibitory effect on tumor size. In some embodiments, the inhibitory effect comprises an inhibitory effect on tumor diameter. In some embodiments, the inhibitory effect comprises an inhibitory effect on tumor width. In some embodiments, the inhibitory effect comprises an inhibitory effect on tumor length. In some embodiments, the inhibitory effect comprises an inhibitory effect on tumor burden. In some embodiments, the inhibitory effect comprises an inhibitory effect on metastasis. In some embodiments, the inhibitory effect comprises an inhibitory effect on an amount of cancer cells.

In some embodiments, the inhibitory effect is 1%, 2.5%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%, or a range of percentages defined by any two of the aforementioned percentages. In some embodiments, the inhibitory effect is about 1%, about 2.5%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98% about 99%, or about 100%, or a range of percentages defined about by any two of the aforementioned percentages. In some embodiments, the inhibitory effect is less than 1%, less than 2.5%, less than 5%, less than less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, less than 96%, less than 97%, less than 98%, less than 99%, or less than 100%. In some embodiments, the inhibitory effect is greater than 1%, greater than 2.5%, greater than 5%, greater than greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90%, greater than 95%, greater than 96%, greater than 97%, greater than 98%, greater than 99%, or greater than 100%.

In some embodiments, the inhibitory effect is relative to a control. In some embodiments, the control is a subject with a cancer or tumor who is untreated. In some embodiments, the control is a subject with a cancer or tumor who is treated with a vehicle. In some embodiments, the control is a subject with a cancer or tumor who is treated with a compound other than an ROR1 inhibitor or antagonist. In some embodiments, the control is a subject without cancer. In some embodiments, the control is a subject without a tumor. In some embodiments, the control is a population or group of such subjects.

In some embodiments, the ROR1 inhibitor or antagonist is formulated as a pharmaceutical composition. In some embodiments, the ROR1 inhibitor or antagonist is formulated in combination with an EGFR inhibitor. In some embodiments, the ROR1 antagonist is formulated for treatment of a subject with a cancer (e.g. a lung cancer such as non-small cell lung cancer). In some embodiments, the ROR1 antagonist is formulated for administration to a subject with cancer in combination with an EGFR inhibitor.

### Therapeutic Methods

EGFR inhibitors and ROR1 antagonists may be used in unison for treating various cancers. These agents may be administered in various dosages, at various schedules, or by various routes, to achieve their anti-cancer effects.

Disclosed herein, in some embodiments, are methods of administering a composition described herein to a subject. For example, some embodiments include administering osimertinib and cirmtuzumab to a subject. Some embodiments relate to use a composition described herein, such as administering the composition to a subject.

Some embodiments relate to a method of treating a disorder such as a cancer or tumor in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder such as a cancer or tumor in the subject. Some embodiments relate to use of a composition described herein in the method of preventing, inhibiting, or reversing the disorder. Some embodiments relate to a method of preventing, inhibiting, or reversing a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing recurrence of a disorder such as a cancer or tumor in a subject in need thereof. Some embodiments relate to use of a composition described herein in the method of preventing recurrence of the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder such as a cancer or tumor in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder such as a cancer or tumor in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In certain embodiments, disclosed herein, are pharmaceutical compositions, EGFR inhibitors, ROR1 antagonists, or combinations thereof useful for the treatment of a cancer or tumor. The pharmaceutical compositions, EGFR inhibitors and ROR1 antagonists can include pharmaceutical compositions, EGFR inhibitors or ROR1 antagonists as described herein. In some such embodiments, the ROR1 antagonist includes an antibody such as cirmtuzumab. In some such embodiments, the EGFR inhibitor includes a small molecule such as osimertinib. Some embodiments of the methods described herein include treatment of a subject. In some embodiments, the subject has a tumor or a cancer. Examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the tumors or cancers express EGFR. In some embodiments, the tumors or cancers express ROR1. In certain embodiments, the tumor is one that has low to moderate levels of Wnt5a signaling or gene expression.

Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will effect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In some embodiments, the tumor or cancer includes a lung cancer. In some embodiments, the lung cancer lung cancer includes a non-small cell lung cancer (NSCLC). In some embodiments, the cancer includes a lymphoma. In some embodiments, the lymphoma includes a mantle cell lymphoma. In some embodiments, the cancer includes a leukemia. In some embodiments, the leukemia includes a chronic lymphocytic leukemia.

In certain embodiments, tumors which can be treated with the EGFR inhibitors or ROR1 antagonists described herein comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor or cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor or cancer to be treated with one or more EGFR inhibitors or ROR1 antagonists described herein comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer (NSCLC), lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the pharmaceutical composition, EGFR inhibitor and/or ROR1 antagonist of this disclosure comprises glioblastoma. In certain embodiments, the cancer treated with the pharmaceutical composition, EGFR inhibitor and/or ROR1 antagonist of this disclosure comprises pancreatic cancer. In certain embodiments, the cancer treated with the pharmaceutical composition, EGFR inhibitor and/or ROR1 antagonist of this disclosure comprises ovarian cancer. In certain embodiments, the cancer treated with the pharmaceutical composition, EGFR inhibitor and/or ROR1 antagonist of this disclosure comprises lung cancer. In certain embodiments, the cancer treated with the pharmaceutical composition, EGFR inhibitor and/or ROR1 antagonist of this disclosure comprises NSCLC. In certain embodiments, the cancer treated with the pharmaceutical composition, EGFR inhibitor and/or ROR1 antagonist of this disclosure comprises leukemia. In certain embodiments, the cancer treated with the pharmaceutical composition, EGFR inhibitor and/or ROR1 antagonist of this disclosure comprises chronic lymphocytic leukemia. In certain embodiments, the cancer treated with the pharmaceutical composition, EGFR inhibitor and/or ROR1 antagonist of this disclosure comprises lymphoma. In certain embodiments, the cancer treated with the pharmaceutical composition, EGFR inhibitor and/or ROR1 antagonist of this disclosure comprises mantle cell lymphoma. In certain embodiments, the cancer treated with the pharmaceutical composition, EGFR inhibitor and/or ROR1 antagonist of this disclosure comprises prostate cancer. In certain embodiments, the cancer treated with the pharmaceutical composition, EGFR inhibitor and/or ROR1 antagonist of this disclosure comprises colon cancer.

In certain embodiments, the cancer treated comprises glioblastoma, pancreatic cancer, ovarian cancer, colon cancer, prostate cancer, or lung cancer (e.g. NSCLC). In a certain embodiment, the cancer is refractory to other treatment. In a certain embodiment, the cancer treated is relapsed. In a certain embodiment, the cancer treated is refractory. In a certain embodiment, the cancer is a relapsed or refractory leukemia (e.g. chronic lymphocytic leukemia), lymphoma (e.g. mantle cell lymphoma), glioblastoma, pancreatic cancer, ovarian cancer, colon cancer, prostate cancer, or lung cancer (e.g. NSCLC). In a certain embodiment, the cancer is a relapsed or refractory lung cancer. In a certain embodiment, the cancer is a relapsed or refractory NSCLC.

The combination of an EGFR inhibitor and an ROR1 antagonist described herein may be for use in treating a cancer wherein said cancer is renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, biliary cancer, or adrenal cancer. In certain embodiments, the cancer is colon adenocarcinoma. In certain embodiments, the cancer is cutaneous melanoma. In certain embodiments, the cancer is glioblastoma multiforme. In certain embodiments, the lung cancer is lung adenocarcinoma. In certain embodiments, the cancer is a non-small cell lung cancer. In certain embodiments, the non-small cell lung cancer comprises a mutation. In certain embodiments, the cancer is a breast cancer. In certain embodiments, the cancer has exhibited resistance to a third-generation EGFR inhibitor as a monotherapy.

In some embodiments, the tumor or cancer comprises a squamous cell carcinoma. In some embodiments, the tumor or cancer comprises a squamous cell carcinoma (SCC) of the lung. In some embodiments, the tumor or cancer comprises a SCC of the lung, and comprises NSCLC. In some embodiments, the tumor or cancer comprises an adenocarcinoma. In some embodiments, the tumor or cancer comprises an adenocarcinoma, and comprises NSCLC. In some embodiments, the tumor or cancer comprises a mesenchymal-epithelial transition factor (MET) amplification. In some embodiments, the tumor or cancer comprises a human epidermal growth factor receptor 2 (HER2) amplification.

In some embodiments, the tumor or cancer comprises an epidermal growth factor receptor (EGFR) mutation. In some embodiments, the EGFR mutation comprises a point mutation or a substitution mutation. In some embodiments, the EGFR mutation comprises an L858 mutation. In some embodiments, the EGFR mutation comprises an L858R mutation. In some embodiments, the L858R mutation increases EGFR activity. In some embodiments, the EGFR mutation comprises a C797 mutation. In some embodiments, the EGFR mutation comprises a C797S mutation. In some embodiments, the EGFR mutation comprises a G796 mutation. In some embodiments, the EGFR mutation comprises a C797 mutation. In some embodiments, the EGFR mutation comprises an L792 mutation. In some embodiments, the EGFR mutation comprises an L718 mutation. In some embodiments, the EGFR mutation comprises an L718Q mutation. In some embodiments, the EGFR mutation comprises a G719 mutation. In some embodiments, the tumor or cancer comprises increased EGFR activity. In some embodiments, the increased EGFR activity is relative to a control or non-cancerous population or subject. In some embodiments, the EGFR mutation comprises a T790 mutation. In some embodiments, the EGFR mutation comprises a T790M mutation. In some embodiments, the T790M mutation confers resistance to first-generation EGFR inhibitors such as erlotinib and gefitinib. In some embodiments, the EGFR mutation comprises an EGFR insertion mutation. In some embodiments, the EGFR insertion mutation comprises an exon-20 insertion. In some embodiments, the EGFR insertion mutation is after a regulatory C-helix of a kinase domain of the EGFR. In some embodiments, the tumor or cancer comprises reduced sensitivity to osimertinib. In some embodiments, the reduced sensitivity to osimertinib is relative to a control or non-cancerous population or subject. In some embodiments, the tumor or cancer comprises reduced sensitivity to a first-generation EGFR inhibitor such as erlotinib or gefitinib. In some embodiments, the reduced sensitivity to the first-generation EGFR inhibitor is relative to a control or non-cancerous population or subject. In some embodiments, the tumor or cancer comprises reduced sensitivity to a second-generation EGFR inhibitor such as afatinib. In some embodiments, the reduced sensitivity to the second-generation EGFR inhibitor is relative to a control or non-cancerous population or subject. In some embodiments, the cancer comprises more than one mutation. In some embodiments, the cancer comprises more than one EGFR mutation. In some embodiments, the cancer comprises 1, 2, 3, 4, 5, 6, 7, 8, 9, or 10, or more mutations or EGFR mutations, or a range of numbers of mutations or EGFR mutations defined by any two of the aforementioned integers.

In certain embodiments, the pharmaceutical composition, EGFR inhibitor and/or ROR1 antagonist can be administered to a subject in need thereof by any route suitable for the administration of EGFR inhibitor- and/or ROR1 antagonist-containing pharmaceutical compositions, such as, for example, a subcutaneous, intraperitoneal, intravenous, intramuscular, intratumoral, or intracerebral, route of administration. In certain embodiments, the pharmaceutical composition, EGFR inhibitor and/or ROR1 antagonist is administered intravenously. In certain embodiments, the pharmaceutical composition, EGFR inhibitor and/or ROR1 antagonist is administered subcutaneously. In certain embodiments, the pharmaceutical composition, EGFR inhibitor and/or ROR1 antagonist is administered intratumorally. In some embodiments, the ROR1 antagonist is administered intravenously. In some embodiments, the EGFR inhibitor is administered intravenously.

In certain embodiments, the pharmaceutical composition, EGFR inhibitor and/or ROR1 antagonist is administered on a suitable dosage schedule, for example, daily, weekly, twice weekly, monthly, twice monthly, once every two weeks, once every three weeks, or once a month. In certain embodiments, the pharmaceutical composition, EGFR inhibitor and/or ROR1 antagonist is administered once every three weeks.

In some embodiments, the EGFR inhibitor is administered once. In some embodiments, the EGFR inhibitor is administered daily. In some embodiments, the EGFR inhibitor is administered once daily. For example, osimertinib may be administered once daily. In some embodiments, the EGFR inhibitor is administered twice daily. In some embodiments, the EGFR inhibitor is administered once every other day. In some embodiments, the EGFR inhibitor is administered twice weekly. In some embodiments, the EGFR inhibitor is administered once weekly. In some embodiments, the EGFR inhibitor is administered once every 10 days. In some embodiments, the EGFR inhibitor is administered once every other week. In some embodiments, the EGFR inhibitor is administered once every 20 days. In some embodiments, the EGFR inhibitor is administered once every three weeks. In some embodiments, the EGFR inhibitor is administered once every 28 days. In some embodiments, the EGFR inhibitor is administered once monthly. In some embodiments, the EGFR inhibitor is administered once every 30 days. In some embodiments, the EGFR inhibitor is administered once every 45 days. In some embodiments, the EGFR inhibitor is administered once every two months. In some embodiments, the EGFR inhibitor is administered once every three months. In some embodiments, the EGFR inhibitor is administered once every 90 days. In some embodiments, the EGFR inhibitor is administered once every four months. In some embodiments, the EGFR inhibitor is administered once every five months. In some embodiments, the EGFR inhibitor is administered once every six months.

In some embodiments, the ROR1 antagonist is administered once. In some embodiments, the ROR1 antagonist is administered daily. In some embodiments, the ROR1 antagonist is administered once daily. In some embodiments, the ROR1 antagonist is administered twice daily. In some embodiments, the ROR1 antagonist is administered once every other day. In some embodiments, the ROR1 antagonist is administered twice weekly. In some embodiments, the ROR1 antagonist is administered once weekly. In some embodiments, the ROR1 antagonist is administered once every 10 days. In some embodiments, the ROR1 antagonist is administered once every other week. For example, osimertinib may be administered once every 14 days. In some embodiments, the ROR1 antagonist is administered once every 20 days. In some embodiments, the ROR1 antagonist is administered once every three weeks. In some embodiments, the ROR1 antagonist is administered once every 28 days. For example, osimertinib may be administered once every 28 days. In some embodiments, the ROR1 antagonist is administered once monthly. In some embodiments, the ROR1 antagonist is administered once every 30 days. In some embodiments, the ROR1 antagonist is administered once every 45 days. In some embodiments, the ROR1 antagonist is administered once every two months. In some embodiments, the ROR1 antagonist is administered once every three months. In some embodiments, the ROR1 antagonist is administered once every 90 days. In some embodiments, the ROR1 antagonist is administered once every four months. In some embodiments, the ROR1 antagonist is administered once every five months. In some embodiments, the ROR1 antagonist is administered once every six months.

In some embodiments, the ROR1 antagonist is administered every 14 days, and then every 28 days. In some embodiments, the ROR1 antagonist is administered every 14 days for 4 doses. In some embodiments, the ROR1 antagonist is administered every 28 days for 4 doses. In some embodiments, the ROR1 antagonist is administered every 14 days for 4 doses, and then every 28 days for 4 doses. In some embodiments, the ROR1 antagonist is again administered every 28 days for an additional 4-6 doses. In some embodiments, the ROR1 antagonist is administered intravenously. In some embodiments, the ROR1 antagonist is cirmtuzumab.

In some embodiments, the pharmaceutical composition, EGFR inhibitor, and/or ROR1 antagonist are administered for a total of 1, 2, 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 weeks, or for a range of time defined by any two of the aforementioned weeks. For example, the pharmaceutical composition, EGFR inhibitor, and/or ROR1 antagonist are administered for 1-56 weeks, 2-24 weeks, or 4-24 weeks.

In some embodiments, the EGFR inhibitor is administered to the subject for at least 1 week. In some embodiments, the EGFR inhibitor is administered to the subject for at least 2 weeks. In some embodiments, the EGFR inhibitor is administered to the subject for at least 3 weeks. In some embodiments, the EGFR inhibitor is administered to the subject for at least 4 weeks. In some embodiments, the EGFR inhibitor is administered to the subject for at least 2 months. In some embodiments, the EGFR inhibitor is administered to the subject for at least 3 months. In some embodiments, the EGFR inhibitor is administered to the subject for at least 4 months. In some embodiments, the EGFR inhibitor is administered to the subject for at least 5 months. In some embodiments, the EGFR inhibitor is administered to the subject for at least 6 months. In some embodiments, the EGFR inhibitor is administered to the subject for at least 1 year. In some embodiments, the EGFR inhibitor is administered to the subject for up to 1 week. In some embodiments, the EGFR inhibitor is administered to the subject for up to 2 weeks. In some embodiments, the EGFR inhibitor is administered to the subject for up to 3 weeks. In some embodiments, the EGFR inhibitor is administered to the subject for up to 4 weeks. In some embodiments, the EGFR inhibitor is administered to the subject for up to 2 months. In some embodiments, the EGFR inhibitor is administered to the subject for up to 3 months. In some embodiments, the EGFR inhibitor is administered to the subject for up to 4 months. In some embodiments, the EGFR inhibitor is administered to the subject for up to 5 months. In some embodiments, the EGFR inhibitor is administered to the subject for up to 6 months. In some embodiments, the EGFR inhibitor is administered to the subject for up to 1 year.

In some embodiments, the ROR1 antagonist is administered to the subject for at least 1 week. In some embodiments, the ROR1 antagonist is administered to the subject for at least 2 weeks. In some embodiments, the ROR1 antagonist is administered to the subject for at least 3 weeks. In some embodiments, the ROR1 antagonist is administered to the subject for at least 4 weeks. In some embodiments, the ROR1 antagonist is administered to the subject for at least 2 months. In some embodiments, the ROR1 antagonist is administered to the subject for at least 3 months. In some embodiments, the ROR1 antagonist is administered to the subject for at least 4 months. In some embodiments, the ROR1 antagonist is administered to the subject for at least 5 months. In some embodiments, the ROR1 antagonist is administered to the subject for at least 6 months. In some embodiments, the ROR1 antagonist is administered to the subject for at least 1 year. In some embodiments, the ROR1 antagonist is administered to the subject for up to 1 week. In some embodiments, the ROR1 antagonist is administered to the subject for up to 2 weeks. In some embodiments, the ROR1 antagonist is administered to the subject for up to 3 weeks. In some embodiments, the ROR1 antagonist is administered to the subject for up to 4 weeks. In some embodiments, the ROR1 antagonist is administered to the subject for up to 2 months. In some embodiments, the ROR1 antagonist is administered to the subject for up to 3 months. In some embodiments, the ROR1 antagonist is administered to the subject for up to 4 months. In some embodiments, the ROR1 antagonist is administered to the subject for up to 5 months. In some embodiments, the ROR1 antagonist is administered to the subject for up to 6 months. In some embodiments, the ROR1 antagonist is administered to the subject for up to 1 year.

In some embodiments, at least 1 dose of the EGFR inhibitor is administered to the subject. In some embodiments, at least 2 doses of the EGFR inhibitor is administered to the subject. In some embodiments, at least 3 doses of the EGFR inhibitor is administered to the subject. In some embodiments, at least 4 doses of the EGFR inhibitor is administered to the subject. In some embodiments, at least 5 doses of the EGFR inhibitor is administered to the subject. In some embodiments, at least 6 doses of the EGFR inhibitor is administered to the subject. In some embodiments, at least 7 doses of the EGFR inhibitor is administered to the subject. In some embodiments, at least 8 doses of the EGFR inhibitor is administered to the subject. In some embodiments, at least 9 doses of the EGFR inhibitor is administered to the subject. In some embodiments, at least 10 doses of the EGFR inhibitor is administered to the subject. In some embodiments, at least 11 doses of the EGFR inhibitor is administered to the subject. In some embodiments, at least 12 doses of the EGFR inhibitor is administered to the subject. In some embodiments, at least 13 doses of the EGFR inhibitor is administered to the subject. In some embodiments, at least 14 doses of the EGFR inhibitor is administered to the subject. In some embodiments, at least 15 doses of the EGFR inhibitor is administered to the subject. In some embodiments, at least 20 doses of the EGFR inhibitor is administered to the subject. In some embodiments, at least 25 doses of the EGFR inhibitor is administered to the subject. In some embodiments, at least 50 doses of the EGFR inhibitor is administered to the subject. In some embodiments, at least 75 doses of the EGFR inhibitor is administered to the subject. In some embodiments, at least 100 doses of the EGFR inhibitor is administered to the subject.

In some embodiments, no more than 1 dose of the EGFR inhibitor is administered to the subject. In some embodiments, no more than 2 doses of the EGFR inhibitor is administered to the subject. In some embodiments, no more than 3 doses of the EGFR inhibitor is administered to the subject. In some embodiments, no more than 4 doses of the EGFR inhibitor is administered to the subject. In some embodiments, no more than 5 doses of the EGFR inhibitor is administered to the subject. In some embodiments, no more than 6 doses of the EGFR inhibitor is administered to the subject. In some embodiments, no more than 7 doses of the EGFR inhibitor is administered to the subject. In some embodiments, no more than 8 doses of the EGFR inhibitor is administered to the subject. In some embodiments, no more than 9 doses of the EGFR inhibitor is administered to the subject. In some embodiments, no more than 10 doses of the EGFR inhibitor is administered to the subject. In some embodiments, no more than 11 doses of the EGFR inhibitor is administered to the subject. In some embodiments, no more than 12 doses of the EGFR inhibitor is administered to the subject. In some embodiments, no more than 13 doses of the EGFR inhibitor is administered to the subject. In some embodiments, no more than 14 doses of the EGFR inhibitor is administered to the subject. In some embodiments, no more than 15 doses of the EGFR inhibitor is administered to the subject. In some embodiments, no more than 20 doses of the EGFR inhibitor is administered to the subject. In some embodiments, no more than 25 doses of the EGFR inhibitor is administered to the subject. In some embodiments, no more than 50 doses of the EGFR inhibitor is administered to the subject. In some embodiments, no more than 75 doses of the EGFR inhibitor is administered to the subject. In some embodiments, no more than 100 doses of the EGFR inhibitor is administered to the subject.

In some embodiments, at least 1 dose of the ROR1 antagonist is administered to the subject. In some embodiments, at least 2 doses of the ROR1 antagonist is administered to the subject. In some embodiments, at least 3 doses of the ROR1 antagonist is administered to the subject. In some embodiments, at least 4 doses of the ROR1 antagonist is administered to the subject. In some embodiments, at least 5 doses of the ROR1 antagonist is administered to the subject. In some embodiments, at least 6 doses of the ROR1 antagonist is administered to the subject. In some embodiments, at least 7 doses of the ROR1 antagonist is administered to the subject. In some embodiments, at least 8 doses of the ROR1 antagonist is administered to the subject. In some embodiments, at least 9 doses of the ROR1 antagonist is administered to the subject. In some embodiments, at least 10 doses of the ROR1 antagonist is administered to the subject. In some embodiments, at least 11 doses of the ROR1 antagonist is administered to the subject. In some embodiments, at least 12 doses of the ROR1 antagonist is administered to the subject. In some embodiments, at least 13 doses of the ROR1 antagonist is administered to the subject. In some embodiments, at least 14 doses of the ROR1 antagonist is administered to the subject. In some embodiments, at least 15 doses of the ROR1 antagonist is administered to the subject. In some embodiments, at least 20 doses of the ROR1 antagonist is administered to the subject. In some embodiments, at least 25 doses of the ROR1 antagonist is administered to the subject. In some embodiments, at least 50 doses of the ROR1 antagonist is administered to the subject. In some embodiments, at least 75 doses of the ROR1 antagonist is administered to the subject. In some embodiments, at least 100 doses of the ROR1 antagonist is administered to the subject.

In some embodiments, no more than 1 dose of the ROR1 antagonist is administered to the subject. In some embodiments, no more than 2 doses of the ROR1 antagonist is administered to the subject. In some embodiments, no more than 3 doses of the ROR1 antagonist is administered to the subject. In some embodiments, no more than 4 doses of the ROR1 antagonist is administered to the subject. In some embodiments, no more than 5 doses of the ROR1 antagonist is administered to the subject. In some embodiments, no more than 6 doses of the ROR1 antagonist is administered to the subject. In some embodiments, no more than 7 doses of the ROR1 antagonist is administered to the subject. In some embodiments, no more than 8 doses of the ROR1 antagonist is administered to the subject. In some embodiments, no more than 9 doses of the ROR1 antagonist is administered to the subject. In some embodiments, no more than 10 doses of the ROR1 antagonist is administered to the subject. In some embodiments, no more than 11 doses of the ROR1 antagonist is administered to the subject. In some embodiments, no more than 12 doses of the ROR1 antagonist is administered to the subject. In some embodiments, no more than 13 doses of the ROR1 antagonist is administered to the subject. In some embodiments, no more than 14 doses of the ROR1 antagonist is administered to the subject. In some embodiments, no more than 15 doses of the ROR1 antagonist is administered to the subject. In some embodiments, no more than 20 doses of the ROR1 antagonist is administered to the subject. In some embodiments, no more than 25 doses of the ROR1 antagonist is administered to the subject. In some embodiments, no more than 50 doses of the ROR1 antagonist is administered to the subject. In some embodiments, no more than 75 doses of the ROR1 antagonist is administered to the subject. In some embodiments, no more than 100 doses of the ROR1 antagonist is administered to the subject.

In some embodiments, the EGFR inhibitor and the ROR1 antagonist are administered simultaneously or sequentially. In some embodiments, the ROR1 antagonist is administered at a first time point and said EGFR inhibitor is administered at a second time point, wherein said first time point precedes said second time point. In some embodiments, the EGFR inhibitor and said ROR1 antagonist are admixed prior to administration. In some embodiments, the EGFR inhibitor and the ROR1 antagonist are administered in a combined synergistic amount.

In some embodiments, the EGFR inhibitor is administered at the same time as the ROR1 antagonist is administered. In some embodiments, the EGFR inhibitor is administered prior to administration of the ROR1 antagonist. In some embodiments, the ROR1 antagonist is administered prior to administration of the EGFR inhibitor. In some embodiments, administration of the EGFR inhibitor begins at the same time as the administration of the ROR1 antagonist. In some embodiments, administration of the EGFR inhibitor begins prior to administration of the ROR1 antagonist. In some embodiments, administration of the ROR1 antagonist begins prior to administration of the EGFR inhibitor. In some embodiments, administration of the EGFR inhibitor ends at the same time as the administration of the ROR1 antagonist. In some embodiments, administration of the EGFR inhibitor ends prior to administration of the ROR1 antagonist. In some embodiments, administration of the ROR1 antagonist ends prior to administration of the EGFR inhibitor. In some embodiments, the EGFR inhibitor is on the same schedule of administration as the ROR1 antagonist. In some embodiments, the EGFR inhibitor is on a different schedule of administration than the ROR1 antagonist.

The pharmaceutical composition, EGFR inhibitor and/or ROR1 antagonist can be administered in any therapeutically effective amount. In certain embodiments, the therapeutically effective amount is between about 0.1 mg/kg and about 50 mg/kg. In certain embodiments, the therapeutically effective amount is between about 1 mg/kg and about 40 mg/kg. In certain embodiments, the therapeutically effective amount is between about 5 mg/kg and about 30 mg/kg. Therapeutically effective amounts include amounts are those sufficient to ameliorate one or more symptoms associated with the disease or affliction to be treated.

In some embodiments, the therapeutically effective amount includes a 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 125 mg/kg, or 150 mg/kg, dose or a range of amounts defined by any of the aforementioned amounts. For example, the EGFR inhibitor may be administered at a dose of 5-100 mg/kg, or the ROR1 antagonist may be administered at a dose of 5-100 mg/kg. In some embodiments, the therapeutically effective amount is a dose or unit dose as described herein.

In some embodiments, the therapeutically effective amount includes an EGFR inhibitor dose. In some embodiments, the EGFR inhibitor dose is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 300 mg, 400 mg, 500 mg, 600 mg, 700 mg, 800 mg, 900 mg, or 1000 mg, or more, or a range of doses defined by any two of the aforementioned doses. For example, the EGFR inhibitor dose may be 1-1000 mg, 1-500 mg, 1-200 mg, 10-150 mg, 25-100 mg, 40-160 mg, about 40-160 mg, 40-80 mg, about 40-80 mg, 80-160 mg, or about 80-160 mg. In some embodiments, the EGFR inhibitor dose is 40 mg. In some embodiments, the EGFR inhibitor dose is 80 mg. For example, a therapeutically effective amount of osimertinib may comprise an 80 mg dose. In some embodiments, the EGFR inhibitor dose is 160 mg.

In some embodiments, the EGFR inhibitor dose is about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 300 mg, about 400 mg, about 500 mg, about 600 mg, about 700 mg, about 800 mg, about 900 mg, or about 1000 mg, or a range of doses defined by any two of the aforementioned doses. In some embodiments, the EGFR inhibitor dose is about 40 mg. In some embodiments, the EGFR inhibitor dose is about 80 mg. In some embodiments, the EGFR inhibitor dose is about 160 mg.

In some embodiments, the EGFR inhibitor dose is at least 1 mg, at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 300 mg, at least 400 mg, at least 500 mg, at least 750 mg, or at least 1000 mg. In some embodiments, the EGFR inhibitor dose is at least 10 mg. In some In some embodiments, the EGFR inhibitor dose is at least 20 mg. In some embodiments, the EGFR inhibitor dose is at least 30 mg. embodiments, the EGFR inhibitor dose is at least 40 mg. In some embodiments, the EGFR inhibitor dose is at least 50 mg. In some embodiments, the EGFR inhibitor dose is at least 60 mg. In some embodiments, the EGFR inhibitor dose is at least 70 mg. In some embodiments, the EGFR inhibitor dose is at least 80 mg. In some embodiments, the EGFR inhibitor dose is at least 90 mg. In some embodiments, the EGFR inhibitor dose is at least 100 mg. In some embodiments, the EGFR inhibitor dose is at least 125 mg. In some embodiments, the EGFR inhibitor dose is at least 150 mg. In some embodiments, the EGFR inhibitor dose is at least 175 mg. In some embodiments, the EGFR inhibitor dose is at least 200 mg. In some embodiments, the EGFR inhibitor dose is at least 300 mg. In some embodiments, the EGFR inhibitor dose is at least 400 mg. In some embodiments, the EGFR inhibitor dose is at least 500 mg. In some embodiments, the EGFR inhibitor dose is at least 750 mg. In some embodiments, the EGFR inhibitor dose is at least 1000 mg.

In some embodiments, the EGFR inhibitor dose is no more than 1 mg, no more than 5 mg, no more than 10 mg, no more than 15 mg, no more than 20 mg, no more than 25 mg, no more than 30 mg, no more than 35 mg, no more than 40 mg, no more than 45 mg, no more than 50 mg, no more than 55 mg, no more than 60 mg, no more than 65 mg, no more than 70 mg, no more than 75 mg, no more than 80 mg, no more than 85 mg, no more than 90 mg, no more than 95 mg, no more than 100 mg, no more than 105 mg, no more than 110 mg, no more than 115 mg, no more than 120 mg, no more than 125 mg, no more than 130 mg, no more than 135 mg, no more than 140 mg, no more than 145 mg, no more than 150 mg, no more than 155 mg, no more than 160 mg, no more than 165 mg, no more than 170 mg, no more than 175 mg, no more than 180 mg, no more than 185 mg, no more than 190 mg, no more than 195 mg, no more than 200 mg, no more than 300 mg, no more than 400 mg, no more than 500 mg, no more than 750 mg, or no more than 1000 mg. In some embodiments, the EGFR inhibitor dose is no more than 10 mg. In some In some embodiments, the EGFR inhibitor dose is no more than 20 mg. In some embodiments, the EGFR inhibitor dose is no more than 30 mg. embodiments, the EGFR inhibitor dose is no more than 40 mg. In some embodiments, the EGFR inhibitor dose is no more than 50 mg. In some embodiments, the EGFR inhibitor dose is no more than 60 mg. In some embodiments, the EGFR inhibitor dose is no more than 70 mg. In some embodiments, the EGFR inhibitor dose is no more than 80 mg. In some embodiments, the EGFR inhibitor dose is no more than 90 mg. In some embodiments, the EGFR inhibitor dose is no more than 100 mg. In some embodiments, the EGFR inhibitor dose is no more than 125 mg. In some embodiments, the EGFR inhibitor dose is no more than 150 mg. In some embodiments, the EGFR inhibitor dose is no more than 175 mg. In some embodiments, the EGFR inhibitor dose is no more than 200 mg. In some embodiments, the EGFR inhibitor dose is no more than 300 mg. In some embodiments, the EGFR inhibitor dose is no more than 400 mg. In some embodiments, the EGFR inhibitor dose is no more than 500 mg. In some embodiments, the EGFR inhibitor dose is no more than 750 mg. In some embodiments, the EGFR inhibitor dose is no more than 1000 mg.

In some embodiments, the EGFR inhibitor dose is below a standard EGFR inhibitor dose (e.g. a manufacturer-suggested dose or an FDA-approved dose of the EGFR inhibitor). For example, a manufacturer-recommended dose of osimertinib is 80 mg (e.g. daily). Thus, some embodiments include administration of an EGFR inhibitor such as osimertinib at a dose below 80 mg to a subject. The EGFR inhibitor dose below a standard EGFR inhibitor dose is particularly advantageous when the subject is also administered a ROR1 antagonist. In some embodiments, the administration of both a third-generation EGFR inhibitor and a ROR1 antagonist to a subject with cancer results in treatment of the cancer at an EGFR inhibitor dose below a standard EGFR inhibitor dose that would not be efficacious at the standard EGFR inhibitor dose. This may benefit the subject by avoiding or decreasing side-effects normally associated with the standard EGFR inhibitor dose. Examples of such side effects include mouth sores; loss of appetite; diarrhea; tiredness; dry skin; rash; or changes to fingernails or toenails such as redness, tenderness, pain, inflammation, brittleness, separation from nailbed, or shedding of nails.

In some embodiments, the EGFR inhibitor is administered at an amount of about from about 20 mg to about 100 mg daily. In some embodiments, the EGFR inhibitor is administered at an amount of about 80 mg daily. In some embodiments, the EGFR inhibitor is administered at an amount of 80 mg daily. In some embodiments, the EGFR inhibitor is administered at an amount of less than about 80 mg daily. In some embodiments, the EGFR inhibitor is administered at an amount of 160 mg daily. In some embodiments, the EGFR inhibitor is administered at an amount of less than about 160 mg daily.

In some embodiments, the therapeutically effective amount is 10 mg/kg. In some embodiments, the therapeutically effective amount is about 10 mg/kg. For example, the therapeutically effective amount of an EGFR inhibitor such as osimertinib may be 10 mg/kg or about 10 mg/kg. Some embodiments include a therapeutically effective amount of an EGFR inhibitor such as osimertinib at 30 mg/kg or about 30 mg/kg. Some embodiments include a therapeutically effective amount of a ROR1 antagonist such as cirmtuzumab at 10 mg/kg or about 10 mg/kg. In some embodiments, the therapeutically effective amount is 15 mg/kg. In some embodiments, the therapeutically effective amount is about 15 mg/kg. For example, the therapeutically effective amount of an EGFR inhibitor such as afatinib may be 15 mg/kg or about 15 mg/kg. In some embodiments, the therapeutically effective amount is 30 mg/kg. In some embodiments, the therapeutically effective amount is about 30 mg/kg. For example, the therapeutically effective amount of an EGFR inhibitor such as rociletinib may be 30 mg/kg or about 30 mg/kg. In some embodiments, the therapeutically effective amount is 40 mg/kg. In some embodiments, the therapeutically effective amount is about 40 mg/kg. For example, the therapeutically effective amount of an EGFR inhibitor such as cetuximab may be 40 mg/kg or about 40 mg/kg. In some embodiments, the therapeutically effective amount is 50 mg/kg. In some embodiments, the therapeutically effective amount is about 50 mg/kg. For example, the therapeutically effective amount of an EGFR inhibitor such as erlotinib may be 50 mg/kg or about 50 mg/kg. In some embodiments, the therapeutically effective amount is 100 mg/kg. In some embodiments, the therapeutically effective amount is about 100 mg/kg. For example, the therapeutically effective amount of an EGFR inhibitor such as gefitinib may be 100 mg/kg or about 100 mg/kg.

In some embodiments, the therapeutically effective amount includes an ROR1 antagonist dose. In some embodiments, the ROR1 antagonist dose is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 300 mg, 400 mg, 500 mg, 600 mg, 700 mg, 800 mg, 900 mg, 1000 mg, 1100 mg, 1200 mg, 1300 mg, 1400 mg, or 1500 mg, or more, or a range of doses defined by any two of the aforementioned doses. For example, the ROR1 antagonist dose may be 1-1500 mg, 100-1000 mg, 250-750 mg, 500-700 mg, about 1-1000 mg, about 250-750 mg, or about 500-700 mg. In some embodiments, the ROR1 antagonist dose is 600 mg. For example, a therapeutically effective amount of cirmtuzumab may comprise an 600 mg dose.

In some embodiments, the ROR1 antagonist dose is about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg, or a range of doses defined by any two of the aforementioned doses. In some embodiments, the ROR1 antagonist dose is about 600 mg.

In some embodiments, the ROR1 antagonist dose is at least 1 mg, at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 300 mg, at least 400 mg, at least 500 mg, at least 600 mg, at least 650 mg, at least 700 mg, at least 750 mg, at least 800, at least 900, at least 1000 mg, at least 1100 mg, at least 1200 mg, at least 1300 mg, at least 1400 mg, or at least 1500 mg. In some embodiments, the ROR1 antagonist dose is at least 10 mg. In some In some embodiments, the ROR1 antagonist dose is at least 20 mg. In some embodiments, the ROR1 antagonist dose is at least 30 mg. embodiments, the ROR1 antagonist dose is at least 40 mg. In some embodiments, the ROR1 antagonist dose is at least 50 mg. In some embodiments, the ROR1 antagonist dose is at least 60 mg. In some embodiments, the ROR1 antagonist dose is at least 70 mg. In some embodiments, the ROR1 antagonist dose is at least 80 mg. In some embodiments, the ROR1 antagonist dose is at least 90 mg. In some embodiments, the ROR1 antagonist dose is at least 100 mg. In some embodiments, the ROR1 antagonist dose is at least 125 mg. In some embodiments, the ROR1 antagonist dose is at least 150 mg. In some embodiments, the ROR1 antagonist dose is at least 175 mg. In some embodiments, the ROR1 antagonist dose is at least 200 mg. In some embodiments, the ROR1 antagonist dose is at least 300 mg. In some embodiments, the ROR1 antagonist dose is at least 400 mg. In some embodiments, the ROR1 antagonist dose is at least 500 mg. In some embodiments, the ROR1 antagonist dose is at least 550 mg. In some embodiments, the ROR1 antagonist dose is at least 600 mg. In some embodiments, the ROR1 antagonist dose is at least 700 mg. In some embodiments, the ROR1 antagonist dose is at least 800 mg. In some embodiments, the ROR1 antagonist dose is at least 900 mg. In some embodiments, the ROR1 antagonist dose is at least 1000 mg. In some embodiments, the ROR1 antagonist dose is at least 1100 mg. In some embodiments, the ROR1 antagonist dose is at least 1200 mg. In some embodiments, the ROR1 antagonist dose is at least 1300 mg. In some embodiments, the ROR1 antagonist dose is at least 1400 mg. In some embodiments, the ROR1 antagonist dose is at least 1500 mg.

In some embodiments, the ROR1 antagonist dose is no more than 1 mg, no more than 5 mg, no more than 10 mg, no more than 15 mg, no more than 20 mg, no more than 25 mg, no more than 30 mg, no more than 35 mg, no more than 40 mg, no more than 45 mg, no more than 50 mg, no more than 55 mg, no more than 60 mg, no more than 65 mg, no more than 70 mg, no more than 75 mg, no more than 80 mg, no more than 85 mg, no more than 90 mg, no more than 95 mg, no more than 100 mg, no more than 105 mg, no more than 110 mg, no more than 115 mg, no more than 120 mg, no more than 125 mg, no more than 130 mg, no more than 135 mg, no more than 140 mg, no more than 145 mg, no more than 150 mg, no more than 155 mg, no more than 160 mg, no more than 165 mg, no more than 170 mg, no more than 175 mg, no more than 180 mg, no more than 185 mg, no more than 190 mg, no more than 195 mg, no more than 200 mg, no more than 300 mg, no more than 400 mg, no more than 500 mg, no more than 600 mg, no more than 650 mg, no more than 700 mg, no more than 750 mg, no more than 800, no more than 900, no more than 1000 mg, no more than 1100 mg, no more than 1200 mg, no more than 1300 mg, no more than 1400 mg, or no more than 1500 mg. In some embodiments, the ROR1 antagonist dose is no more than 10 mg. In some In some embodiments, the ROR1 antagonist dose is no more than 20 mg. In some embodiments, the ROR1 antagonist dose is no more than 30 mg. embodiments, the ROR1 antagonist dose is no more than 40 mg. In some embodiments, the ROR1 antagonist dose is no more than 50 mg. In some embodiments, the ROR1 antagonist dose is no more than 60 mg. In some embodiments, the ROR1 antagonist dose is no more than 70 mg. In some embodiments, the ROR1 antagonist dose is no more than 80 mg. In some embodiments, the ROR1 antagonist dose is no more than 90 mg. In some embodiments, the ROR1 antagonist dose is no more than 100 mg. In some embodiments, the ROR1 antagonist dose is no more than 125 mg. In some embodiments, the ROR1 antagonist dose is no more than 150 mg. In some embodiments, the ROR1 antagonist dose is no more than 175 mg. In some embodiments, the ROR1 antagonist dose is no more than 200 mg. In some embodiments, the ROR1 antagonist dose is no more than 300 mg. In some embodiments, the ROR1 antagonist dose is no more than 400 mg. In some embodiments, the ROR1 antagonist dose is no more than 500 mg. In some embodiments, the ROR1 antagonist dose is no more than 550 mg. In some embodiments, the ROR1 antagonist dose is no more than 600 mg. In some embodiments, the ROR1 antagonist dose is no more than 700 mg. In some embodiments, the ROR1 antagonist dose is no more than 800 mg. In some embodiments, the ROR1 antagonist dose is no more than 900 mg. In some embodiments, the ROR1 antagonist dose is no more than 1000 mg. In some embodiments, the ROR1 antagonist dose is no more than 1100 mg. In some embodiments, the ROR1 antagonist dose is no more than 1200 mg. In some embodiments, the ROR1 antagonist dose is no more than 1300 mg. In some embodiments, the ROR1 antagonist dose is no more than 1400 mg. In some embodiments, the ROR1 antagonist dose is at least 1500 mg.

In some embodiments, the ROR1 antagonist dose is below a standard ROR1 antagonist dose (e.g. a manufacturer-suggested dose or an FDA-approved dose of the ROR1 antagonist). For example, a dose of cirmtuzumab that may be recommended is 600 mg (e.g. every two or four weeks). Thus, some embodiments include administration of a ROR1 antagonist such as cirmtuzumab at a dose below 600 mg to a subject. The ROR1 antagonist dose below a standard ROR1 antagonist dose is particularly advantageous when the subject is also administered an EGFR inhibitor. In some embodiments, the administration of both a third-generation EGFR inhibitor and a ROR1 antagonist to a subject with cancer results in treatment of the cancer at a ROR1 antagonist dose below a standard ROR1 antagonist dose that would not be efficacious at a standard ROR1 antagonist dose. This may benefit the subject by avoiding or decreasing side-effects normally associated with a standard ROR1 antagonist dose.

In some embodiments, the combined treatment of administering and EGFR inhibitor and a ROR1 antagonist is nontoxic. In some embodiments, the combined treatment of administering and EGFR inhibitor and a ROR1 antagonist prevents or decreases the amount or severity of one or more adverse effects. In some embodiments, the combined treatment prevents or decreases toxicity. In some embodiments, the combined treatment prevents or decreases weight loss. In some embodiments, the combined treatment prevents or decreases one or more grade 1 adverse effects (e.g., mild; asymptomatic or mild symptoms; clinical or diagnostic observations only; or where no intervention is indicated). In some embodiments, the combined treatment prevents or decreases one or more grade 2 adverse effects (e.g., moderate; minimal, where local or noninvasive intervention is indicated; or limiting age-appropriate instrumental activities of daily living). In some embodiments, the combined treatment prevents or decreases one or more grade 3 adverse effects (e.g., severe or medically significant but not immediately life-threatening; where hospitalization or prolongation of hospitalization is indicated; disabling; or limiting self-care activities of daily living), one or more grade 4 adverse effects (e.g., life-threatening consequences; or where urgent intervention is indicated). In some embodiments, the combined treatment prevents or decreases death.

In some embodiments, the ROR1 antagonist dose is 2 mg/kg, 4 mg/kg, 8 mg/kg, or 16 mg/kg, for example, 2 mg/kg, 4 mg/kg, 8 mg/kg, or 16 mg/kg of cirmtuzumab. In some embodiments, the ROR1 antagonist dose is 2 mg/kg. In some embodiments, the ROR1 antagonist dose is 4 mg/kg. In some embodiments, the ROR1 antagonist dose is 8 mg/kg. In some embodiments, the ROR1 antagonist dose is 16 mg/kg.

In some embodiments, the ROR1 antagonist is administered at an amount of about from about 100 mg to about 1000 mg every other week or monthly. In some embodiments, the ROR1 antagonist is administered at an amount of about 600 mg every other week. In some embodiments, the ROR1 antagonist is administered at an amount of 600 mg every other week. In some embodiments, the ROR1 antagonist is administered at an amount of less than about 600 mg every other week. In some embodiments, the ROR1 antagonist is administered at an amount of about 600 mg monthly. In some embodiments, the ROR1 antagonist is administered at an amount of 600 mg monthly. In some embodiments, the ROR1 antagonist is administered at an amount of less than about 600 mg monthly.

In some embodiments, the ROR1 inhibitor or antagonist has an inhibitory effect such as inhibiting ROR1 activity, inhibiting the expression of an ROR1 protein, increasing the degradation of an ROR1 protein, or inhibiting the expression of an ROR1 transcript. In some embodiments, the inhibitory effect comprises an inhibitory effect on cell growth. In some embodiments, the inhibitory effect comprises an inhibitory effect on cell division. In some embodiments, the inhibitory effect comprises an increase in cell death. In some embodiments, the cell death comprises apoptosis. In some embodiments, the inhibitory effect comprises an inhibitory effect on tumor growth. In some embodiments, the inhibitory effect comprises an inhibitory effect on tumor volume. In some embodiments, the inhibitory effect comprises an inhibitory effect on tumor size. In some embodiments, the inhibitory effect comprises an inhibitory effect on tumor diameter. In some embodiments, the inhibitory effect comprises an inhibitory effect on tumor width. In some embodiments, the inhibitory effect comprises an inhibitory effect on tumor length. In some embodiments, the inhibitory effect comprises an inhibitory effect on tumor burden. In some embodiments, the inhibitory effect comprises an inhibitory effect on metastasis. In some embodiments, the inhibitory effect comprises an inhibitory effect on an amount of cancer cells.

In some embodiments, the administration may result in a treatment effect on the cancer or tumor. For example, the treatment effect may comprise a reduction in tumor volume, a reduction in tumor size, a decrease in metastasis, or a decrease in an amount of cancer cells in the subject as compared to prior to treatment. In some embodiments, the treatment effect is observed or occurs within 1 week, within 2 weeks, within 3 weeks, within 1 month, within 2 months, within 3 months, within 4 months, within 5 months, within 6 months, within 7 months, within 8 months, within 9 months, within 10 months, within 11 months, within 1 year, within 2 years, within 3 years, within 4 years, or within 5 years after initiation of treatment with the pharmaceutical composition, EGFR inhibitor, ROR1 antagonist. In some embodiments, the treatment effect is observed or occurs after 1 week, after 2 weeks, after 3 weeks, after 1 month, after 2 months, after 3 months, after 4 months, after 5 months, after 6 months, after 7 months, after 8 months, after 9 months, after 10 months, after 11 months, after 1 year, after 2 years, after 3 years, after 4 years, or after 5 years after initiation of treatment with the pharmaceutical composition, EGFR inhibitor, ROR1 antagonist. For example, tumor volume may be reduced within 2 weeks after the first dose.

In some embodiments, the treatment effect is observed within 1 week of administration of the EGFR inhibitor. In some embodiments, the treatment effect is observed within 2 weeks of administration of the EGFR inhibitor. In some embodiments, the treatment effect is observed within 3 weeks of administration of the EGFR inhibitor. In some embodiments, the treatment effect is observed within 1 month of administration of the EGFR inhibitor. In some embodiments, the treatment effect is observed within 2 months of administration of the EGFR inhibitor. In some embodiments, the treatment effect is observed within 3 months of administration of the EGFR inhibitor. In some embodiments, the treatment effect is observed within 4 months of administration of the EGFR inhibitor. In some embodiments, the treatment effect is observed within 5 months of administration of the EGFR inhibitor. In some embodiments, the treatment effect is observed within 6 months of administration of the EGFR inhibitor. In some embodiments, the treatment effect is observed within 1 year of administration of the EGFR inhibitor.

In some embodiments, the treatment effect is observed within 1 week of administration of the ROR1 antagonist. In some embodiments, the treatment effect is observed within 2 weeks of administration of the ROR1 antagonist. In some embodiments, the treatment effect is observed within 3 weeks of administration of the ROR1 antagonist. In some embodiments, the treatment effect is observed within 1 month of administration of the ROR1 antagonist. In some embodiments, the treatment effect is observed within 2 months of administration of the ROR1 antagonist. In some embodiments, the treatment effect is observed within 3 months of administration of the ROR1 antagonist. In some embodiments, the treatment effect is observed within 4 months of administration of the ROR1 antagonist. In some embodiments, the treatment effect is observed within 5 months of administration of the ROR1 antagonist. In some embodiments, the treatment effect is observed within 6 months of administration of the ROR1 antagonist. In some embodiments, the treatment effect is observed within 1 year of administration of the ROR1 antagonist.

Some embodiments of the methods described herein include obtaining a baseline measurement from a subject. For example, in some embodiments, a baseline measurement is obtained from the subject prior to treating the subject.

In some embodiments, the baseline measurement is a baseline tumor volume measurement. In some embodiments, the baseline measurement is a baseline tumor size measurement. In some embodiments, the baseline measurement is a baseline tumor width measurement. In some embodiments, the baseline measurement is a baseline tumor length measurement. In some embodiments, the baseline measurement is a baseline cancer burden. In some embodiments, the baseline measurement is a baseline number of tumors. In some embodiments, the baseline measurement is a baseline number of cancer cells. In some embodiments, the baseline measurement is a baseline tumor growth rate. In some embodiments, the baseline measurement is the presence of cancer.

In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, or a fluorescence assay. In some embodiments, the baseline measurement is obtained by PCR. In some embodiments, the baseline measurement is obtained by a medical imaging device. In some embodiments, the baseline measurement is obtained by ultrasound. In some embodiments, the baseline measurement is obtained by magnetic resonance imaging (MRI). In some embodiments, the baseline measurement is obtained by a functional MRI (fMRI). In some embodiments, the baseline measurement is obtained by positron emission tomography (PET). In some embodiments, the baseline measurement is obtained visually. In some embodiments, the baseline measurement is obtained through use of a microscope. In some embodiments, the baseline measurement is obtained by a histologic measurement. In some embodiments, the baseline measurement is obtained in a biopsy. In some embodiments, the baseline measurement is obtained in a blood sample. In some embodiments, the baseline measurement is obtained directly from the patient.

In some embodiments, administration of the pharmaceutical composition, EGFR inhibitor, ROR1 antagonist affects a measurement such as a tumor volume measurement, tumor size measurement, tumor width measurement, tumor length measurement, cancer burden, number of tumors, number of cancer cells, tumor growth rate, or presence of cancer, relative to the baseline measurement. In some embodiments, the administration reduces the tumor volume relative to the baseline tumor volume measurement. In some embodiments, the administration reduces the tumor size measurement relative to the tumor size measurement. In some embodiments, the administration reduces the tumor width measurement relative to the baseline tumor width measurement. In some embodiments, the administration reduces the tumor length measurement relative to the baseline tumor length measurement. In some embodiments, the administration reduces the cancer burden measurement relative to the baseline cancer burden measurement. In some embodiments, the administration reduces the number of tumors relative to the baseline number of tumors. In some embodiments, the administration reduces the number of cancer cells relative to the baseline number of cancer cells. In some embodiments, the administration reduces the tumor growth rate relative to the baseline tumor growth rate. In some embodiments, the administration abolishes the presence of cancer. In some embodiments, the measurement is reduced by 2.5%, 5%, 7.5%, 19%, 20%, 30%, 40%, 50%, 60%, 70%, 80%, 90%, 100%, 200% 300%, 400%, 500%, 600%, 700%, 800%, 900%, or 1000%, or by a range defined by any of the two aforementioned percentages, relative to the baseline measurement.

In some embodiments, the measurement is obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated. In some embodiments, the measurement is obtained by performing an assay such as an immunoassay, a colorimetric assay, or a fluorescence assay, on the sample obtained from the subject. In some embodiments, the measurement is obtained by an immunoassay, a colorimetric assay, or a fluorescence assay. In some embodiments, the measurement is obtained by PCR. In some embodiments, the measurement is obtained by a medical imaging device. In some embodiments, the measurement is obtained by ultrasound. In some embodiments, the measurement is obtained by magnetic resonance imaging (MRI). In some embodiments, the measurement is obtained by a functional MRI (fMRI). In some embodiments, the measurement is obtained by positron emission tomography (PET). In some embodiments, the measurement is obtained visually. In some embodiments, the measurement is obtained through use of a microscope. In some embodiments, the measurement is obtained by a histologic measurement. In some embodiments, the measurement is obtained in a biopsy. In some embodiments, the measurement is obtained in a blood sample. In some embodiments, the measurement is obtained directly from the patient.

Some embodiments include use of a composition described herein in a method of treating a subject with a cancer, wherein the subject has already been treated with or administered a ROR1 antagonist. Some such methods may include administering a third-generation EGFR inhibitor to a subject who has already been receiving treatment with the ROR1 antagonist for 1 month, 2 months, 3 months, 4 months, 5 months, 6 months, 7 months, 8 months, 9 months, 10 months, 11 months, 12 months, or longer, prior to receiving treatment with the third-generation EGFR inhibitor. Some such methods may include administering a third-generation EGFR inhibitor to a subject 1 month, 2 months, 3 months, 4 months, 5 months, 6 months, 7 months, 8 months, 9 months, 10 months, 11 months, 12 months, or longer, after induction of therapy with a ROR1 antagonist, or after an administration of a ROR1 antagonist to the subject. Some embodiments include use of a composition comprising a third-generation EGFR inhibitor such osimertinib in a method of treating a cancer such as lung cancer in a subject, wherein the subject has already been treated with a ROR1 antagonist such as cirmtuzumab.

Some embodiments include use of a composition described herein in a method of treating a subject with a cancer, wherein the subject has already been treated with or administered an EGFR inhibitor. Some such methods may include administering a ROR1 antagonist to a subject who has already been receiving treatment with a third-generation EGFR inhibitor for 1 month, 2 months, 3 months, 4 months, 5 months, 6 months, 7 months, 8 months, 9 months, 10 months, 11 months, 12 months, or longer, prior to receiving treatment with the ROR1 antagonist. Some such methods may include administering a ROR1 antagonist to a subject 1 month, 2 months, 3 months, 4 months, 5 months, 6 months, 7 months, 8 months, 9 months, 10 months, 11 months, 12 months, or longer, after induction of therapy with a third-generation EGFR inhibitor, or after an administration of a third-generation EGFR inhibitor to the subject. Some embodiments include use of a composition comprising a ROR1 antagonist such as cirmtuzumab in a method of treating a cancer such as lung cancer in a subject, wherein the subject has already been treated with an third-generation EGFR inhibitor such osimertinib.

Some embodiments relate to a method of modulating a cancer cell signaling pathway, comprising administering an EGFR inhibitor and a ROR1 antagonist to a subject with cancer. The method may include use of an EGFR inhibitor, a ROR1 antagonist, a pharmaceutical composition, or a method such as a dose or treatment schedule as described herein. In some embodiments, the administration prevents or reduces ROR1 activation in a cancer cell. The reduction or prevention of ROR1 activation may prevent or decrease metastasis or tumor growth in the subject. In some embodiments, the administration prevents or decreases GEF activation. The reduction or prevention of GEF activation may prevent or decrease metastasis or tumor growth in the subject. In some embodiments, the administration prevents or decreases activity of a GTPase. The reduction or prevention of the GTPase activity may prevent or decrease metastasis or tumor growth in the subject. In some embodiments, the administration prevents or decreases Rac1 activity in a cancer cell. The reduction or prevention of Rac1 activity may prevent or decrease tumor growth in the subject. In some embodiments, the administration prevents or reduces RhoA activity in a cancer cell. The reduction or prevention of RhoA activity may prevent or decrease metastasis in the subject. In some embodiments, the administration prevents or reduces ROCK activation in a cancer cell. The reduction or prevention of ROCK activation may prevent or decrease metastasis in the subject. In some embodiments, the administration prevents or reduces EGFR activation in a cancer cell. The reduction or prevention of EGFR activation may prevent or decrease metastasis or tumor growth in the subject. In some embodiments, the administration prevents or reduces a downstream EGFR signaling pathway, such as a Ras pathway or a PI3K pathway, in a cancer cell. The reduction or prevention of EGFR activation may prevent or decrease metastasis or tumor growth in the subject.

### Patient Selection

It may be useful to provide the combined cancer treatment with an EGFR inhibitor and a ROR1 antagonist to some patients after determining that an alternative form of treatment is likely to be ineffective, or after confirming the alternative's ineffectiveness. Additionally, the combined cancer treatment may work best in certain patient populations. Accordingly, some methods are useful in indicating which patients to administer the combined treatment to.

Disclosed herein, in some embodiments, are methods of selecting a subject for treatment. The treatment may comprise a therapeutic method as described herein, such as a method of treating cancer in the subject by administering an EGFR inhibitor and a ROR1 antagonist to the subject as described. In some embodiments, the subject has cancer. In some embodiments, the subject has a lung cancer (e.g. non-small cell lung cancer).

Some embodiments include administering to the subject a therapeutically effective amount of an EGFR inhibitor and a ROR1 antagonist, provided a presence of a cancer phenotype is detected in a sample obtained from the subject. In some embodiments, the administration reduces the cancer phenotype.

Some embodiments include contacting a sample obtained from a subject comprising genetic material with an assay adapted to detect a presence of a cancer phenotype. Some embodiments include (selecting the subject for treatment with an EGFR inhibitor and a ROR1 antagonist, provided the presence of the cancer phenotype is detected. Some embodiments include a method of selecting a subject for treatment with an EGFR inhibitor and a ROR1 antagonist, the method comprising: (a) contacting a sample obtained from a subject comprising genetic material with an assay adapted to detect a presence of a cancer phenotype; and (b) selecting the subject for treatment with an EGFR inhibitor and a ROR1 antagonist, provided the presence of the cancer phenotype is detected in (a).

Some embodiments include determining whether a subject is, or is at risk for developing, non-response or loss-of-response to a standard therapy. Some embodiments include determining whether the subject is suitable for treatment an EGFR inhibitor and a ROR1 antagonist. Some embodiments include contacting a sample obtained from the subject with an assay adapted to detect a presence of a cancer phenotype. In some embodiments, determining whether the subject is suitable for treatment an EGFR inhibitor and a ROR1 antagonist includes contacting a sample obtained from the subject with an assay adapted to detect a presence of a cancer phenotype. Some embodiments include detecting the cancer phenotype in the sample obtained from the subject. In some embodiments, determining whether the subject is suitable for treatment an EGFR inhibitor and a ROR1 antagonist includes detecting the cancer phenotype in the sample obtained from the subject.

Some embodiments include: if the subject is not determined to have, or be at risk for developing, the non-response or loss-of-response to the standard therapy, then treating the subject by administering a therapeutically effective amount of the standard therapy to the subject. Some embodiments include: if the subject is determined to have, or be at risk for developing, the non-response or loss-of-response to the standard therapy, and the subject is determined to be suitable for treatment with the EGFR inhibitor and the ROR1 antagonist, then treating the subject by administering a therapeutically effective amount of the EGFR inhibitor and the ROR1 antagonist to the subject. Some embodiments include treating the subject by administering a therapeutically effective amount of the EGFR inhibitor and the ROR1 antagonist to the subject when the subject is determined to have, or be at risk for developing, the non-response or loss-of-response to the standard therapy, and when the subject is determined to be suitable for treatment with the EGFR inhibitor and the ROR1 antagonist; and treating the subject by administering a therapeutically effective amount of the standard therapy to the subject when the subject is not determined to have, or be at risk for developing, the non-response or loss-of-response to the standard therapy, and/or is not determined to be suitable for treatment with the EGFR inhibitor and the ROR1 antagonist.

Some embodiments include a method of treating a tumor or cancer in a subject, the method comprising: (a) determining whether a subject is, or is at risk for developing, non-response or loss-of-response to a standard therapy; (b) determining whether the subject is suitable for treatment an EGFR inhibitor and a ROR1 antagonist by a process of: (i) contacting a sample obtained from the subject with an assay adapted to detect a presence of a cancer phenotype, and (ii) detecting the cancer phenotype in the sample obtained from the subject; (c) if the subject is not determined to have, or be at risk for developing, the non-response or loss-of-response to the standard therapy, then treating the subject by administering a therapeutically effective amount of the standard therapy to the subject; and (d) if the subject is determined to have, or be at risk for developing, the non-response or loss-of-response to the standard therapy, and the subject is determined to be suitable for treatment with the EGFR inhibitor and the ROR1 antagonist, then treating the subject by administering a therapeutically effective amount of the EGFR inhibitor and the ROR1 antagonist to the subject.

In some embodiments, the cancer phenotype comprises non-responsiveness to a standard therapy. In some embodiments, the cancer phenotype comprises non-responsiveness to an EGFR inhibitor. In some embodiments, the cancer phenotype comprises non-responsiveness to an EGFR inhibitor alone. In some embodiments, the cancer phenotype comprises non-responsiveness to an EGFR inhibitor in combination with another therapy other than a ROR1 antagonist. In some embodiments, the cancer phenotype comprises non-responsiveness to an EGFR inhibitor in combination with another therapy other than cirmtuzumab. In some embodiments, the EGFR inhibitor comprises a first-generation EGFR inhibitor. In some embodiments, the EGFR inhibitor comprises a second-generation EGFR inhibitor. In some embodiments, the EGFR inhibitor comprises a third-generation EGFR inhibitor. In some embodiments, the EGFR inhibitor comprises a third-generation EGFR inhibitor other than cirmtuzumab. In some embodiments, the cancer phenotype comprises non-responsiveness to any cancer therapy other than a combination of an EGFR inhibitor and a ROR1 antagonist. In some embodiments, the cancer phenotype comprises non-responsiveness to an EGFR inhibitor and/or a ROR1 antagonist other than a combination of osimertinib and cirmtuzumab. In some embodiments, the cancer phenotype comprises non-responsiveness to any cancer therapy other than a combination of osimertinib and cirmtuzumab. In some embodiments, non-responsiveness includes lack of an improved phenotype over a treatment period. In some embodiments, the cancer phenotype includes a lack of an optimal response to therapy. For example, the cancer phenotype may include a minimal response to afatinib therapy alone or in combination with a ROR1 antagonist.

In some embodiments, the cancer phenotype comprises a cancer genotype. In some embodiments, the cancer genotype comprises a mutation that confers resistance to a cancer treatment. In some embodiments, the cancer genotype comprises a mutation that confers resistance to an EGFR inhibitor. For example, the cancer genotype may include an EGFR T790 mutation. In some embodiments, the cancer genotype comprises an EGFR T790M mutation. In some embodiments, the cancer genotype comprises a mutation that confers resistance to a first-generation EGFR inhibitor. In some embodiments, the cancer genotype comprises a mutation that confers resistance to a second-generation EGFR inhibitor. In some embodiments, the cancer genotype comprises a mutation that confers resistance to a third-generation EGFR inhibitor. In some embodiments, the cancer genotype comprises a mutation that increases EGFR activity. For example, the cancer genotype may include an EGFR L858 mutation. In some embodiments, the cancer genotype comprises an EGFR L858R mutation. In some embodiments, the cancer genotype comprises an EGFR C797 mutation. In some embodiments, the cancer genotype comprises an EGFR C797S mutation. In some embodiments, the cancer genotype comprises an EGFR G796 mutation. In some embodiments, the cancer genotype comprises an EGFR C797 mutation. In some embodiments, the cancer genotype comprises an EGFR L792 mutation. In some embodiments, the cancer genotype comprises an EGFR L718 mutation. In some embodiments, the cancer genotype comprises an EGFR L718Q mutation. In some embodiments, the cancer genotype comprises an EGFR G719 mutation. In some embodiments, the cancer genotype comprises an exon 19 deletion. In some embodiments, the cancer genotype comprises an exon 21 mutation. In some embodiments, administration of the EGFR and/or ROR1 antagonist is indicated for treatment of patients with metastatic non-small cell lung cancer (NSCLC) whose tumors have epidermal growth factor receptor (EGFR) exon 19 deletions or exon 21 L858R mutations. In some embodiments, administration of the EGFR and/or ROR1 antagonist is indicated for metastatic EGFR T790M mutation positive NSCLC, in patients who have progressed on or after EGFR tyrosine kinase inhibitor therapy. In some embodiments, the cancer genotype comprises more than one mutation or EGFR mutation. In some embodiments, the cancer genotype comprises 1, 2, 3, 4, 5, 6, 7, 8, 9, or 10, or more EGFR mutations, or a range of numbers of EGFR mutations defined by any two of the aforementioned integers.

In some embodiments, the cancer phenotype comprises an EGFR expression level. In some embodiments, the cancer phenotype comprises a EGFR activity level. In some embodiments, the EGFR activity or expression level is in relation to a control. In some embodiments, the EGFR activity or expression is increased in relation to the control. In some embodiments, the EGFR activity or expression is decreased in relation to the control. In some embodiments, the EGFR expression comprises an EGFR mRNA expression level. In some embodiments, the EGFR expression comprises an EGFR protein expression level. In some embodiments, the cancer genotype comprises an EGFR mutation.

In some embodiments, the cancer phenotype comprises a ROR1 expression level. In some embodiments, the cancer phenotype comprises a ROR1 activity level. In some embodiments, the ROR1 activity or expression level is in relation to a control. In some embodiments, the ROR1 activity or expression is increased in relation to the control. In some embodiments, the ROR1 activity or expression is decreased in relation to the control. In some embodiments, the ROR1 expression comprises a ROR1 mRNA expression level. In some embodiments, the ROR1 expression comprises a ROR1 protein expression level. In some embodiments, the cancer genotype comprises a ROR1 mutation.

In some embodiments, the cancer phenotype comprises a WNT5a expression level. In some embodiments, the WNT5a expression level is in relation to a control. In some embodiments, the WNT5a expression is increased in relation to the control. In some embodiments, the WNT5a expression is decreased in relation to the control. In some embodiments, the WNT5a expression comprises a WNT5a mRNA expression level. In some embodiments, the WNT5a expression comprises a WNT5a protein expression level. In some embodiments, the cancer genotype comprises a WNT5a mutation.

In some embodiments, the cancer phenotype comprises GEF activation. In some embodiments, the cancer phenotype comprises an activity of a GTPase. In some embodiments, the cancer phenotype comprises Rac1 activation. In some embodiments, the cancer phenotype comprises RhoA activation. In some embodiments, the cancer phenotype comprises ROCK activation. In some embodiments, the cancer phenotype comprises activation of a Ras pathway. In some embodiments, the cancer phenotype comprises activation of a PI3K pathway. In some embodiments, the cancer phenotype comprises cMet amplification.

In some embodiments, the control includes a noncancerous sample from the subject with cancer or at risk of having cancer. In some embodiments, the control is a sample from a healthy subject without cancer. In some embodiments, the control is a sample from a population without cancer. In some embodiments, the control is a cancer sample from a subject that is not resistant to a cancer therapy.

In some embodiments, the cancer phenotype comprises stage I non-small cell lung cancer (NSCLC). In some embodiments, the cancer phenotype comprises stage II NSCLC. In some embodiments, the cancer phenotype comprises stage IIIA NSCLC. In some embodiments, the cancer phenotype comprises N2 lymph nodes. In some embodiments, the cancer phenotype comprises stage IIIB NSCLC. In some embodiments, the cancer phenotype comprises stage IV NSCLC. In some embodiments, the cancer phenotype comprises an inoperable phenotype.

In some embodiments, the assay adapted to detect a presence of a cancer phenotype comprises polymerase chain reaction (PCR), quantitative reverse-transcription PCR (qPCR), automated sequencing, genotype array, or a combination thereof. Methods disclosed herein for detecting a cancer phenotype in a sample from a subject comprise analyzing the genetic material in the sample to detect at least one of a presence, an absence, and a quantity of a nucleic acid sequence encompassing the cancer phenotype. In some cases, the nucleic acid sequence comprises DNA. In some instances, the nucleic acid sequence comprises RNA. In some instances, the nucleic acid comprises an RNA transcript.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next-generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a cancer phenotype provided herein.

In some embodiments, detecting the presence or absence of a cancer phenotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some embodiments, the standard therapy comprises a surgery. For example, radical surgery may be the standard therapy for a subject with stage I non-small cell lung cancer (NSCLC). In some embodiments, the standard therapy comprises chemotherapy. In some embodiments, the chemotherapy comprises cisplatin chemotherapy. In some embodiments, the cisplatin chemotherapy comprises 4 cycles of cisplatin chemotherapy. For example, cisplatin chemotherapy may be the standard therapy for a subject with stage II or IIIA NSCLC. In some embodiments, the standard therapy comprises radiotherapy. For example, radiotherapy may be the standard therapy for subjects with N2 lymph nodes. In some embodiments, the standard therapy comprises an EGFR inhibitor. In some embodiments, the standard therapy comprises a first-generation EGFR inhibitor. In some embodiments, the standard therapy comprises a second-generation EGFR inhibitor. In some embodiments, the standard therapy comprises a third-generation EGFR inhibitor. In some embodiments, the standard therapy comprises cisplatin chemotherapy and a third-generation EGFR inhibitor. For example, in patients with stage IIIB/IV or inoperable NSCLC, the standard therapy may include cisplatin chemotherapy and a third-generation EGFR inhibitor. In some embodiments, the standard therapy does not include an EGFR inhibitor. In some embodiments, the standard therapy does not include a second-generation EGFR inhibitor. In some embodiments, the standard therapy does not include a third-generation EGFR inhibitor. In some embodiments, the standard therapy does not include a ROR1 antagonist. In some embodiments, the standard therapy does not include osimertinib. In some embodiments, the standard therapy does not include cirmtuzumab.

In one aspect described herein is a method of treating a cancer in an individual comprising assaying a sample from the individual for an EGFR mutation and administering a combination of an ROR1 antagonist and a third-generation EGFR inhibitor. In certain embodiments, the mutation comprises a substitution at one or more of L718, G719, L792, C797, L858 of the EGFR protein or a gene encoding the EGFR protein. In certain embodiments, the mutation comprises a one or more of a deletion or insertion of exon 19 or Exon 20 of the EGFR gene. In certain embodiments, the mutation comprises a one or more of a deletion or insertion of exon 19 or Exon 20 of the EGFR gene. In certain embodiments, the sample may be a blood sample or a tumor biopsy.

In one aspect described herein is a method of treating a cancer in an individual comprising assaying a sample from the individual for an EGFR mutation and administering a combination of cirmtuzumab and a third-generation EGFR inhibitor. In certain embodiments, the mutation comprises a substitution at one or more of L718, G719, L792, C797, L858 of the EGFR protein or a gene encoding the EGFR protein. In certain embodiments, the mutation comprises a one or more of a deletion or insertion of exon 19 or Exon 20 of the EGFR gene. In certain embodiments, the mutation comprises a one or more of a deletion or insertion of exon 19 or Exon 20 of the EGFR gene. In certain embodiments, the sample may be a blood sample or a tumor biopsy.

In one aspect described herein is a method of treating a cancer in an individual comprising assaying a sample from the individual for an EGFR mutation and administering a combination of cirmtuzumab and osimertinib. In certain embodiments, the mutation comprises a substitution at one or more of L718, G719, L792, C797, L858 of the EGFR protein or a gene encoding the EGFR protein. In certain embodiments, the mutation comprises a one or more of a deletion or insertion of exon 19 or Exon 20 of the EGFR gene. In certain embodiments, the sample may be a blood sample or a tumor biopsy.

The methods described herein may be used to treat patients already treated with a third-generation EGFR inhibitor, or patients that have developed resistance to third-generation EGFR inhibitors. In certain embodiments, resistance is characterized by progressive disease despite third-generation EGFR inhibitor treatment.

### Pharmaceutically Acceptable Excipients, Carriers, and Diluents

It may be advantageous to administer the EGFR inhibitors and ROR1 antagonists in separate or combined pharmaceutical formulations. For example, various carriers, excipients and diluents may aid in administering the drugs in therapeutically significant doses.

In certain embodiments an EGFR inhibitor and/or ROR1 antagonist of the current disclosure is included in a pharmaceutical composition comprising one or more pharmaceutically acceptable excipients, carriers, and diluents. In certain embodiments, an EGFR inhibitor and/or ROR1 antagonist of the current disclosure is administered suspended in a sterile solution. Some embodiments include pharmaceutical compositions comprising an EGFR inhibitor, a ROR1 antagonist, and an excipient, carrier or adjuvant.

In certain embodiments, the solution comprises NaCl. In certain embodiments, the solution comprises about 0.9% NaCl. In certain embodiments, the solution comprises dextrose. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the EGFR inhibitor and/or ROR1 antagonist of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The EGFR inhibitor and/or ROR1 antagonist, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

In some embodiments, the pharmaceutical composition contains at least one excipient. In some embodiments, the excipient is an antiadherent, a binder, a coating, a color or dye, a disintegrant, a flavor, a glidant, a lubricant, a preservative, a sorbent, a sweetener, or a vehicle. In some embodiments, the excipient comprises a wetting or emulsifying agent, or a pH buffering agent. In some embodiments, the excipient contains pharmaceutically acceptable salts to adjust the osmotic pressure, buffers, preservatives and the like.

In some embodiments, the pharmaceutical composition contains at least one pharmaceutically acceptable carrier. In some embodiments, the carrier is saline, buffered saline, dextrose, water, glycerol, sesame oil, ethanol, and combinations thereof. In some embodiments, the pharmaceutically acceptable carrier is determined in part by the particular pharmaceutical composition being administered, and/or by the particular method used to administer the pharmaceutical composition. Pharmaceutically acceptable carriers include, but are not limited to, saline, buffered saline, dextrose, water, glycerol, sesame oil, ethanol, and combinations thereof. In some embodiments, the carrier is sterile, and the formulation suits the mode of administration. In some embodiments, the pharmaceutical composition contains a liquid solution, suspension, emulsion, tablet, pill, capsule, sustained release formulation, or powder.

Some embodiments include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, comprising saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. In some embodiments, preservatives or other additives are present such as, for example, antimicrobials, anti-oxidants, chelating agents, and inert gases and the like. In some embodiments, the carrier comprises one or more biodegradable, mucoadhesive polymeric carriers. In some embodiments, the excipient or carrier comprises one or more hydrophilic polymers, such as sodium alginate or carbopol.

In some embodiments, the carrier comprises a liquid solution, suspension, emulsion, tablet, pill, capsule, sustained release formulation, or powder. In some embodiments, the pharmaceutical composition comprises a liquid, or a lyophilized or freeze-dried powder. In some embodiments, the pharmaceutical composition is formulated as a suppository, with traditional binders and carriers such as triglycerides. In some embodiments, oral formulations include one or more standard carriers such as pharmaceutical grades of mannitol, lactose, starch, magnesium stearate, sodium saccharine, cellulose, and magnesium carbonate.

In some embodiments, the pharmaceutical composition comprises a vehicle comprising 0.5% methocellulose-0.4% Tween 80 in water. In some embodiments, the pharmaceutical composition comprises a vehicle comprising 5% DMSO, 15% Solutol HS15, and 80% water. In some embodiments, the pharmaceutical composition comprises a vehicle comprising 5% DMSO, 30% PEG300 and 65% water. In some embodiments, the pharmaceutical composition comprises a vehicle comprising 1% CMC Na in water.

Also described herein are kits comprising a ROR1 antibody and an EGFR inhibitor or antagonist in a suitable container and one or more additional components selected from: instructions for use; a diluent, an excipient, a carrier, and a device for administration. In some embodiments, the device for administration comprises a needle.

In some embodiments, the pharmaceutical composition is formulated for needle administration. In some embodiments, the pharmaceutical composition is formulated for intravenous administration. In some embodiments, the pharmaceutical composition is formulated for oral administration. In some embodiments, the pharmaceutical composition is formulated for intranasal, intradermal, intramuscular, topical, oral, subcutaneous, intraperitoneal, intravenous, or intrathecal administration.

In some embodiments, the pharmaceutical composition comprises a dose of 1 μL, 10 μL, 50 μL, 100 μL, 250 μL, 500 μL, 750 μL, 1 mL, 1.25 mL, 1.5 mL, 1.75 mL, 2 mL, 2.5 mL, 3 mL, 3.5 mL, 4 mL, 4.5 mL, or 5 mL of the pharmaceutical composition, or a range of doses defined by any two of the aforementioned doses. In some embodiments, the pharmaceutical composition comprises a dose of 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, or 2.5 g of the pharmaceutical composition, or a range of doses defined by any two of the aforementioned doses. In some embodiments, the pharmaceutical composition comprises a dose of 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1 g, 1.1 g, 1.2 g, 1.3 g, 1.4 g, 1.5 g, 1.6 g, 1.7 g, 1.8 g, 1.9 g, 2.0 g, 2.1 g, 2.2 g, 2.3 g, 2.4 g, or 2.5 g of the EGFR inhibitor and/or the ROR1 inhibitor, or a range of doses defined by any two of the aforementioned doses.

In some embodiments, the pharmaceutical composition comprises a unit dose. In some embodiments, the unit dose comprises a unit dose of the EGFR inhibitor. In some embodiments, the unit dose comprises a unit dose of the ROR1 antagonist. In some embodiments, the unit dose comprises a unit dose of the EGFR inhibitor and the ROR1 antagonist combined. In some embodiments, the unit dose comprises a therapeutically effective amount of a composition as described herein. For example, a unit dose of osimertinib may be 40 mg, about 40 mg, 80 mg, or about 80 mg of osimertinib; or a unit dose of cirmtuzumab may be 600 mg or about 600 mg of cirmtuzumab.

Some embodiments include a method of manufacturing a composition comprising an EGFR inhibitor described herein and a ROR1 antagonist described herein for use in a method described herein such as a method of treatment. Some embodiments include manufacturing said composition. Some embodiments include a method comprising manufacturing a third-generation EGFR inhibitor for use in a method described herein, wherein the subject has already been treated with a ROR1 antagonist. Some embodiments include a method of manufacturing a composition comprising a third-generation EGFR inhibitor such osimertinib for use in a method of treating a cancer such as lung cancer in a subject, wherein the subject has already been treated with a ROR1 antagonist such as cirmtuzumab. Some embodiments include a method comprising manufacturing a ROR1 antagonist for use in a method described herein, wherein the subject has already been treated with a third-generation EGFR inhibitor. Some embodiments include a method of manufacturing a composition comprising a ROR1 antagonist such as cirmtuzumab for use in a method of treating a cancer such as lung cancer in a subject, wherein the subject has already been treated with an third-generation EGFR inhibitor such osimertinib.

### Embodiments

Some embodiments include one or more of the following:
1. A method of treating cancer in a subject in need thereof, said method comprising administering to said subject a therapeutically effective amount of an epidermal growth factor receptor (EGFR) inhibitor and a tyrosine kinase-like orphan receptor 1 (ROR1) antagonist.
2. The method of embodiment 1, wherein said EGFR inhibitor is a small molecule.
3. The method of embodiment 1 or 2, wherein said EGFR inhibitor is a third-generation EGFR inhibitor such as osimertinib, AC0010, lapatinib, mavelertinib, naquotinib, nazartinib, olmutinib, or rociletinib.
4. The method of any one of embodiments 1-3, wherein said EGFR inhibitor is osimertinib.
5. The method of any one of embodiments 1-4, wherein said ROR1 antagonist is an antibody or a small molecule.
6. The method of any one of embodiments 1-5, wherein said ROR1 antagonist is an anti-ROR1 antibody.
7. The method of embodiment 5 or 6, wherein said antibody comprises a Fab, F(ab′)₂, Fv, or an scFv.
8. The method of any one of embodiments 5-7, wherein said antibody comprises a heavy chain variable region and a light chain variable region, wherein said heavy chain variable region comprises an amino acid sequence at least about 85%, 90%, 95%, 97%, 98%, 99%, or 100 identical to that set forth in SEQ ID NO: 7; and wherein said light chain variable region comprises an amino acid sequence at least about 85%, 90%, 95%, 97%, 98%, 99%, or 100 identical to that set forth in SEQ ID NO: 8.
9. The method of any one of embodiments 5-8, wherein said antibody comprises a humanized heavy chain variable region and a humanized light chain variable region, wherein said humanized heavy chain variable region comprises the sequences set forth in SEQ ID NO:1, SEQ ID NO:2, and SEQ ID NO:3; and wherein said humanized light chain variable region comprises the sequences set forth in SEQ ID NO:4, SEQ ID NO:5, and SEQ ID NO:6.
10. The method of any one of embodiments 5-9, wherein said antibody is cirmtuzumab.
11. The method of any one of embodiments 1-10, wherein said individual is afflicted with a cancer that comprises a mutated EGFR gene.
12. The method of embodiment 11, wherein the mutated EGFR gene comprises a mutation resulting in a T790M mutation or an L858R mutation in the EGFR protein or an exon-20 insertion in the EGFR gene.
13. The method of any one of embodiments 1-12, wherein said EGFR inhibitor and said ROR1 antagonist are administered in a combined synergistic amount.
14. The method of any one of embodiments 1-13, wherein said EGFR inhibitor and said ROR1 antagonist are administered simultaneously or sequentially.
15. The method of any one of embodiments 1-14, wherein said ROR1 antagonist is administered at a first time point and said EGFR inhibitor is administered at a second time point, wherein said first time point precedes said second time point.
16. The method of any one of embodiments 1-14, wherein said EGFR inhibitor and said ROR1 antagonist are admixed prior to administration.
17. The method of any one of embodiments 1-16, wherein said EGFR inhibitor is administered at an amount of about from about 20 mg to about 100 mg daily.
18. The method of any one of embodiments 1-17, wherein said EGFR inhibitor is administered at an amount of about 80 mg daily.
19. The method of any one of embodiments 1-17, wherein said EGFR inhibitor is administered at an amount of less than about 80 mg daily.
20. The method of any one of embodiments 1-19, wherein said EGFR inhibitor is administered intravenously.
21. The method of any one of embodiments 1-20, wherein said ROR1 antagonist is administered intravenously.
22. The method of any one of embodiments 1-21, wherein said subject is a mammal.
23. The method of any one of embodiments 1-22, wherein said subject is a human.
24. The method of any one of embodiments 1-23, wherein said cancer is renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, or adrenal cancer.
25. The method of any one of embodiments 1-24 wherein the cancer is a non-small cell lung cancer.
26. The method of embodiment 25, wherein the non-small cell lung cancer comprises a mutation resulting in a T790M mutation or an L858R mutation in the EGFR protein or an exon-20 insertion in the EGFR gene.
27. The method of any one of embodiments 1-24, wherein said cancer comprises a mutation resulting in a T790M mutation or an L858R mutation in the EGFR protein or an exon-20 insertion in the EGFR gene.
28. A pharmaceutical composition comprising an EGFR inhibitor, a ROR1 antagonist and a pharmaceutically acceptable excipient.
29. The pharmaceutical composition of embodiment 28, wherein the EGFR inhibitor comprises osimertinib and the ROR1 antagonist comprises cirmtuzumab.
30. The pharmaceutical composition of embodiment 28 or 29, comprising a unit dosage of the EGFR inhibitor and the ROR1 antagonist.
31. Use of a composition comprising an EGFR inhibitor and a ROR1 antagonist in a method of treating cancer.
32. The use of embodiment 31, wherein the composition further comprises a pharmaceutically acceptable excipient.
33. The use of embodiment 31 or 32, wherein the composition comprises the pharmaceutical composition of any one of embodiments 28-30.
34. The use of any one of embodiments 31-33, wherein the method comprises administering to a subject in need of cancer treatment, or suspected to be of need of cancer treatment, a therapeutically effective amount of the composition.
35. The use of any one of embodiments 31-34, wherein said EGFR inhibitor is a small molecule.
36. The use of any one of embodiments 31-35, wherein said EGFR inhibitor comprises osimertinib, afatinib, cetuximab, dacomitinib, erlotinib, gefitinib, lapatinib, necitumumab, neratinib, panitumumab, rociletinib, or vandetanib.
37. The use of any one of embodiments 31-36, wherein said EGFR inhibitor is erlotinib, gefitinib, afatinib, or osimertinib.
38. The use of any one of embodiments 31-37, wherein the EGFR inhibitor comprises a third-generation EGFR inhibitor.
39. The use of any one of embodiments 31-38, wherein the third-generation EGFR inhibitor comprises lapatinib, osimertinib or rociletinib.
40. The use of any one of embodiments 31-39, wherein said EGFR inhibitor is osimertinib.
41. The use of any one of embodiments 31-40, wherein said ROR1 antagonist is an antibody or a small molecule.
42. The use of any one of embodiments 31-41, wherein said ROR1 antagonist is an anti-ROR1 antibody.
43. The use of embodiment 41 or 42, wherein said antibody comprises a Fab, F(ab′)₂, Fv, or an scFv.
44. The use of any one of embodiments 41-43, wherein said antibody comprises a heavy chain variable region and a light chain variable region, wherein said heavy chain variable region comprises an amino acid sequence at least about 85%, 90%, 95%, 97%, 98%, 99%, or 100 identical to that set forth in SEQ ID NO: 7; and wherein said light chain variable region comprises an amino acid sequence at least about 85%, 90%, 95%, 97%, 98%, 99%, or 100 identical to that set forth in SEQ ID NO: 8.
45. The use of any one of embodiments 41-44, wherein said antibody comprises a humanized heavy chain variable region and a humanized light chain variable region, wherein said humanized heavy chain variable region comprises the sequences set forth in SEQ ID NO:1, SEQ ID NO:2, and SEQ ID NO:3; and wherein said humanized light chain variable region comprises the sequences set forth in SEQ ID NO:4, SEQ ID NO:5, and SEQ ID NO:6.
46. The use of any one of embodiments 41-45, wherein said antibody is cirmtuzumab.
47. The use of any one of embodiments 31-46, wherein said individual is afflicted with a cancer that comprises a mutated EGFR gene.
48. The use of embodiment 47, wherein the mutated EGFR gene comprises a mutation resulting in a T790M mutation or an L858R mutation in the EGFR protein or an exon-20 insertion in the EGFR gene.
49. The use of any one of embodiments 31-48, wherein said EGFR inhibitor and said ROR1 antagonist are administered in a combined synergistic amount.
50. The use of any one of embodiments 31-49, wherein said EGFR inhibitor and said ROR1 antagonist are administered simultaneously or sequentially.
51. The use of any one of embodiments 31-50, wherein said ROR1 antagonist is administered at a first time point and said EGFR inhibitor is administered at a second time point, wherein said first time point precedes said second time point.
52. The use of any one of embodiments 31-51, wherein said EGFR inhibitor and said ROR1 antagonist are admixed prior to administration.
53. The use of any one of embodiments 31-52, wherein said EGFR inhibitor is administered at an amount of about from about 20 mg to about 100 mg daily.
54. The use of any one of embodiments 31-53, wherein said EGFR inhibitor is administered at an amount of about 80 mg daily.
55. The use of any one of embodiments 31-53, wherein said EGFR inhibitor is administered at an amount of less than about 80 mg daily.
56. The use of any one of embodiments 31-55, wherein said EGFR inhibitor is administered intravenously.
57. The use of any one of embodiments 31-56, wherein said ROR1 antagonist is administered intravenously.
58. The use of any one of embodiments 31-57, wherein said subject is a mammal.
59. The use of any one of embodiments 31-58, wherein said subject is a human.
60. The use of any one of embodiments 31-59, wherein said cancer is lymphoma, leukemia, myeloma, AML, B-ALL, T-ALL, renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, or adrenal cancer.
61. The use of any one of embodiments 31-60 wherein the cancer is a lung cancer.
62. The use of any one of embodiments 31-61 wherein the cancer is a non-small cell lung cancer.
63. The use of embodiment 62, wherein the non-small cell lung cancer comprises a mutation resulting in a T790M mutation or an L858R mutation in the EGFR protein or an exon-20 insertion in the EGFR gene.
64. The use of any one of embodiments 31-60, wherein said cancer is chronic lymphocytic leukemia (CLL), small lymphocytic lymphoma, marginal cell B-Cell lymphoma, Burkitt's Lymphoma, or B cell leukemia.
65. Use of a composition comprising a third-generation EGFR inhibitor such osimertinib in a method of treating a cancer such as lung cancer in a subject, wherein the subject has already been treated with a ROR1 antagonist such as cirmtuzumab.
66. Use of a composition comprising a ROR1 antagonist such as cirmtuzumab in a method of treating a cancer such as lung cancer in a subject, wherein the subject has already been treated with an third-generation EGFR inhibitor such osimertinib.
67. A method of manufacturing a composition comprising an EGFR inhibitor such osimertinib and a ROR1 antagonist such as cirmtuzumab for use in a method of treating a cancer such as lung cancer.
68. A method of manufacturing a composition comprising a third-generation EGFR inhibitor such osimertinib for use in a method of treating a cancer such as lung cancer in a subject, wherein the subject has already been treated with a ROR1 antagonist such as cirmtuzumab.
69. A method of manufacturing a composition comprising a ROR1 antagonist such as cirmtuzumab for use in a method of treating a cancer such as lung cancer in a subject, wherein the subject has already been treated with an third-generation EGFR inhibitor such osimertinib.

### EXAMPLES

The following illustrative examples are representative of embodiments of compositions and methods described herein and are not meant to be limiting in any way.

### Example 1—EGFR Inhibitor and ROR1 Antagonism Increases Treatment Efficacy in a Mouse Model of NSCLC

The anti-tumor activity of the tyrosine kinase-like orphan receptor 1 (ROR1) antagonist, cirmtuzumab, was tested in combination with the third-generation epidermal growth factor receptor inhibitor (EGFRi), osimertinib, in a patient-derived xenograft (PDX) mouse model of non-small cell lung cancer (NSCLC).

### LU PDX Cell Lines

Experiments were performed using the LU0858 cell line, which is MET amplified and has an EGFR L858R mutation. The L858R mutation increases EGFR activity in LU0858. When cMET is inhibited in LU0858, EGFRi sensitivity is restored. LU0858 expresses ROR1, and shows reduced sensitivity to osimertinib when delivered alone (Table 1).

Some proposed experiments include use of the LU3075 cell line, which harbors EGFR exon-20 insertions after a regulatory C-helix of a kinase domain, and responds poorly to known EGFR inhibitors. Like LU0858, LU3075 expresses ROR1, and shows reduced sensitivity to osimertinib.

TABLE 1

| | LU0858 | LU3075 |
|---|---|---|
| ROR1 expression (log2) (FPKM) | 1.9669 | 1.3192 |
| Wnt5a expression (log2) (FPKM) | −2.0000 | 1.4359 |
| Growth kinetics (20 days) | 250 −> 1500 mm² | 150 −> 500 mm² |
| Osimertinib resistance | 25-60 mg/kg PR | 30 mg/kg PR |
| | | (maybe 10 mg/kg |
| | | PR) |

### Tumor Inoculation

Fresh tumor tissues from mice bearing established primary human lung cancer patient-derived xenograft (PDX) model LU0858 were harvested and cut into small pieces (approximately 2-3 mm in diameter). PDX tumor fragments, harvested from donor mice, were inoculated subcutaneously at the upper right dorsal flank into female BALB/c nude mice for tumor development.

### Observation and Data Collection

After tumor inoculation, the animals were checked daily for morbidity and mortality. During routine monitoring, the animals are checked for any effects of tumor growth and treatments on behavior such as mobility, food and water consumption, body weight gain/loss (body weights were measured twice per week after randomization or based on a sponsor's request after randomization), eye/hair matting and any other abnormalities. Mortality and observed clinical signs were recorded for individual animals in detail. Tumor volumes were measured twice per week in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L).

### Study Design and Results

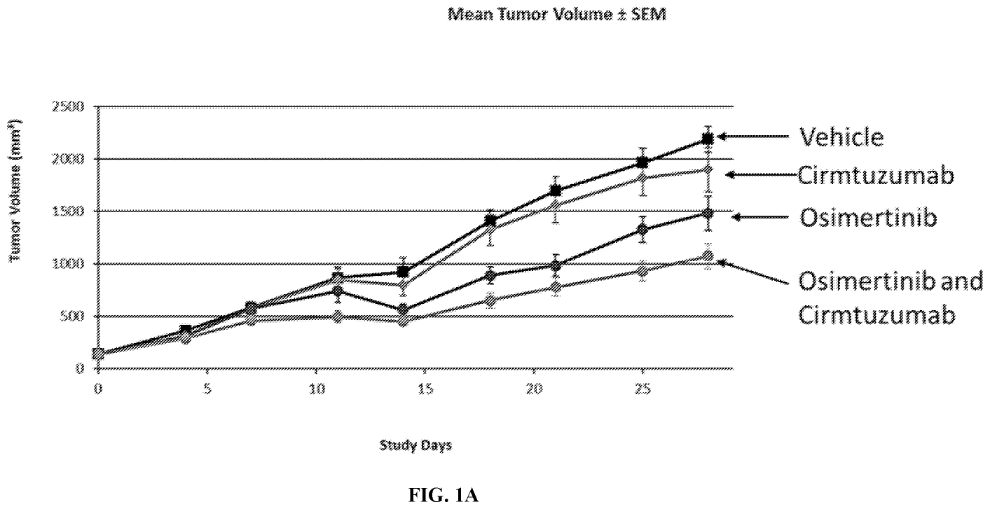

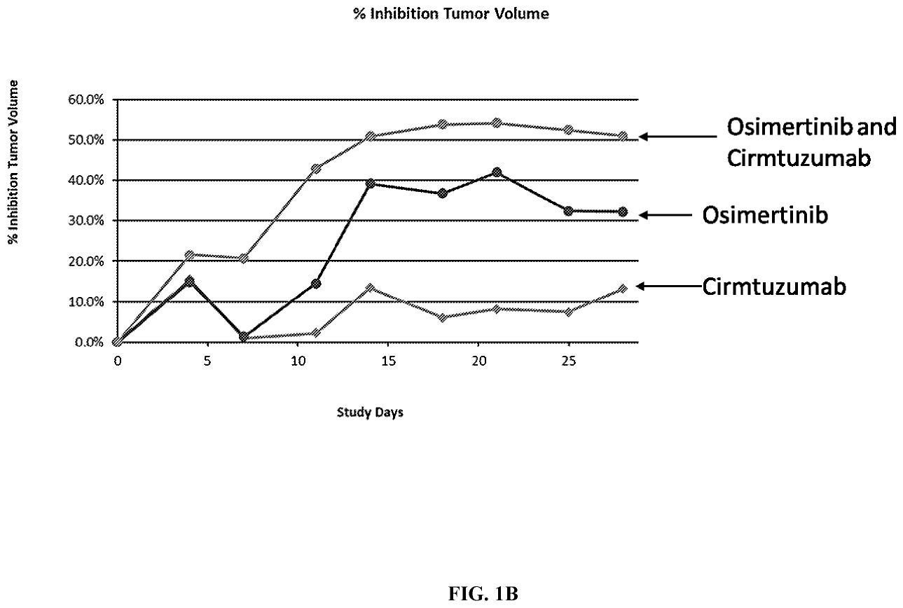

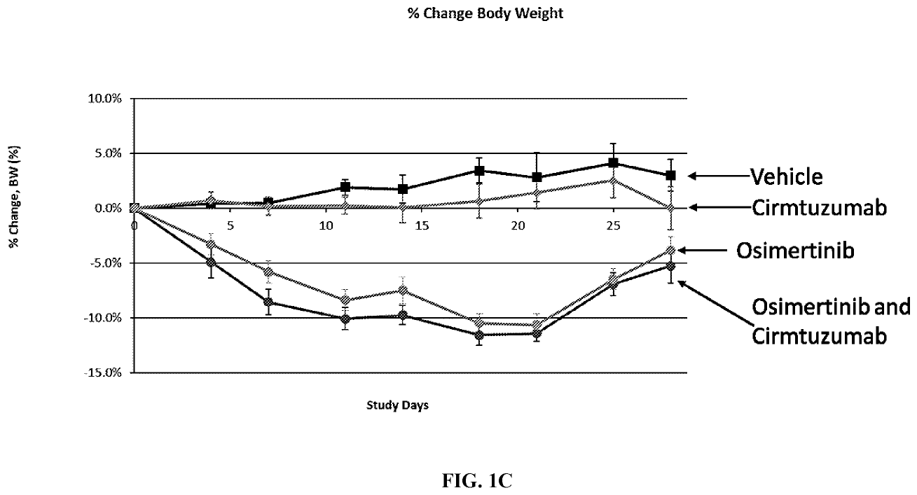

Table 2 shows the study design that was executed for the LU0858 model. In the LU0858 model, the combination of cirmtuzumab (UC-961) and osimertinib showed a strong synergistic inhibition of tumor growth FIG. 1A and FIG. 1B, and Table 4, and was highly tolerated (e.g., less toxic) as shown in FIG. 1C. These data indicate that third-generation EGFRi such as osimertinib show a surprisingly effective synergistic anti-cancer effect when combined with osimertinib.

TABLE 2
Study design LU0858 Model

| | | | | Dosing | | Dosing |
|---|---|---|---|---|---|---|
| | Animal | | Dose level | Volume | | Frequency & |
| Group | No. | Treatment | (mg/kg) | (μL/g) | ROA | Duration |
| 1 | 8 | Vehicle | — | 10 | p.o. | QD × 21 days |
| 2 | 8 | UC-961 | 10 | 10 | i.v | QW × 4 |
| 3 | 8 | Osimertinib | 30 | 10 | p.o. | QD × 21 days |
| 4 | 8 | UC-961 | 10 | 10 | i.v. | QW × 4 |
| | | Osimertinib | 30 | 10 | p.o. | QD × 21 days |

Table 3 shows a proposed study design for the LU3075 model. In the LU3075 model, the combination of cirmtuzumab and osimertinib is expected to synergistically inhibit tumor growth and be highly tolerated at either of two doses of osimertinib.

TABLE 3
Study design LU3075 Model

| | | | | Dosing | | Dosing |
|---|---|---|---|---|---|---|
| | Animal | | Dose level | Volume | | Frequency & |
| Group | No. | Treatment | (mg/kg) | (μL/g) | ROA | Duration |
| 1 | 8 | Vehicle | — | 10 | p.o. | QD × 21 days |
| 2 | 8 | UC-961 | 10 | 10 | i.v. | QW × 4 |
| 3 | 8 | Osimertinib | 7.5 | 10 | p.o. | QD × 21 days |
| 4 | 8 | UC-961 | 10 | 10 | i.v. | QW × 4 |
| | | Osimertinib | 7.5 | 10 | p.o. | QD × 21 days |
| 5 | 8 | Osimertinib | 20 | 10 | p.o. | QD × 21 days |
| 6 | 8 | UC-961 | 10 | 10 | i.v. | QW × 4 |
| | | Osimertinib | 20 | 10 | p.o. | QD × 21 days |

TABLE 4
Efficacy of treatment in LU0858 cells

| | | On day 21 | | | On day 28 | | |
|---|---|---|---|---|---|---|---|
| | | Tumor Size | TGI | P | Tumor Size | TGI | P |
| Group | Treatment Description | (mm³) | (%) | Value | (mm³) | (%) | Value |
| 1 | Vehicle, 0 mg/kg, QD × 21 days, | 1696.5 ± 137.1 | — | — | 2189.4 ± 123.6 | — | — |
| | p.o. | | | | | | |
| 2 | UC961, 10 mg/kg, QW × 4, i.v. | 1558.8 ± 167.2 | 8.1 | >0.05 | 1899.3 ± 209.5 | 13.3 | >0.05 |
| 3 | Osimertinib, 30 mg/kg, QD × 21 | 985.0 ± 99.4 | 41.9 | <0.01 | 1483.9 ± 162.2 | 32.2 | <0.05 |
| | days, p.o. | | | | | | |
| 4 | UC961, 10 mg/kg, QW × 4, i.v., | 777.6 ± 86.6 | 54.2 | <0.001 | 1073.8 ± 119.5 | 51.0 | <0.001 |
| | Osimertinib, 30 mg/kg, QD × 21 | | | | | | |
| | days, p.o. | | | | | | |

TGI = tumor growth inhibition

### Example 2—First- and Second-Generation EGFR Inhibitors Combined with Cirmtuzumab are Ineffective in a Mouse Model of NSCLC

The anti-tumor activity of cirmtuzumab was tested in combination with first and second-generation epidermal growth factor receptor inhibitors (EGFRi) in a cell line xenograft mouse model of non-small cell lung cancer (NSCLC).

### NCI-H1975 Cell Line

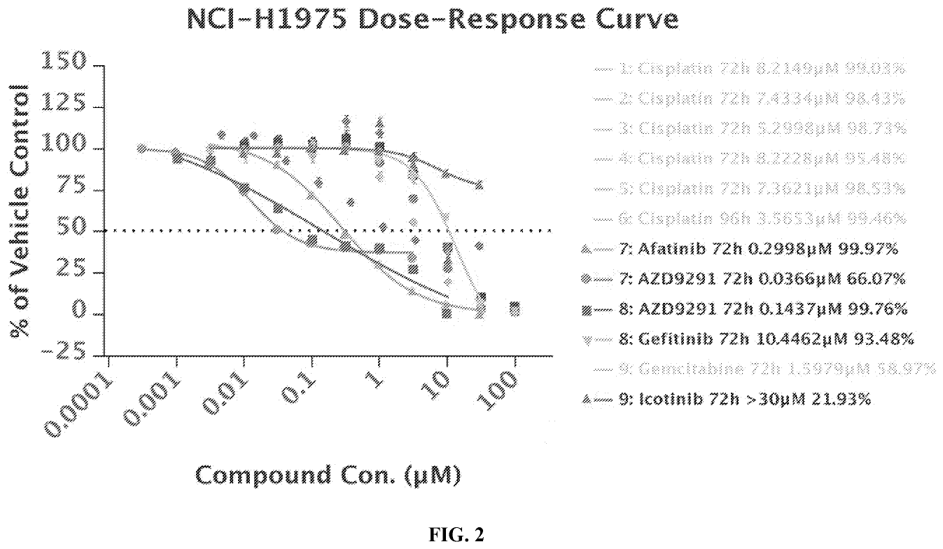

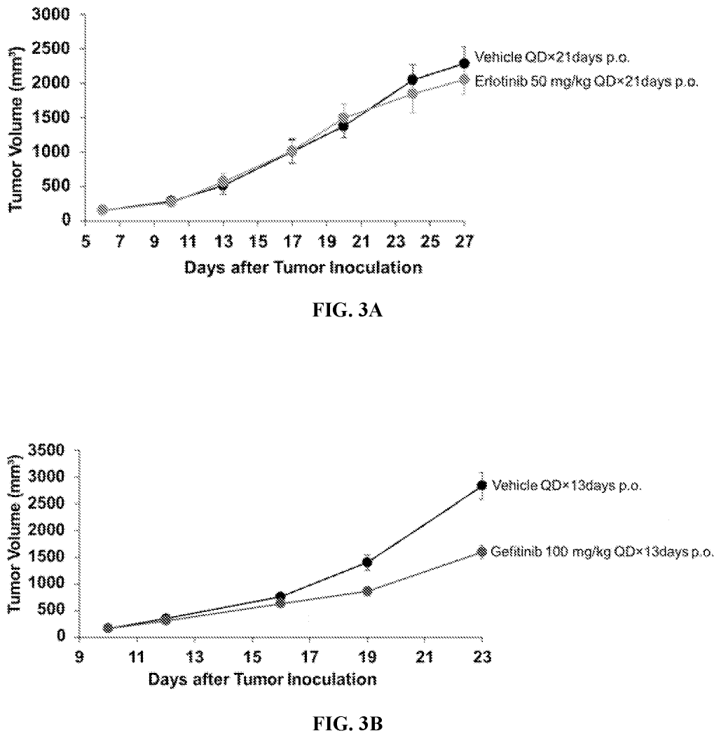

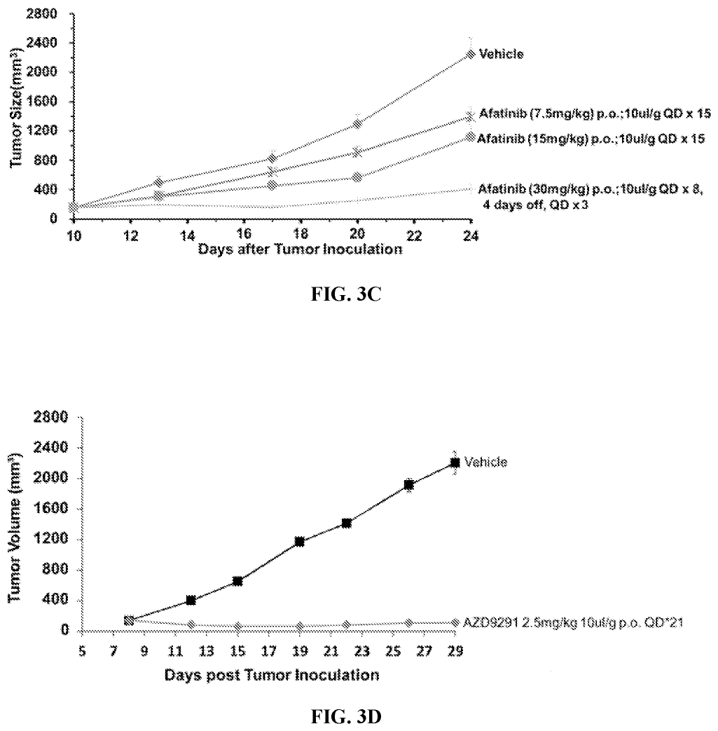

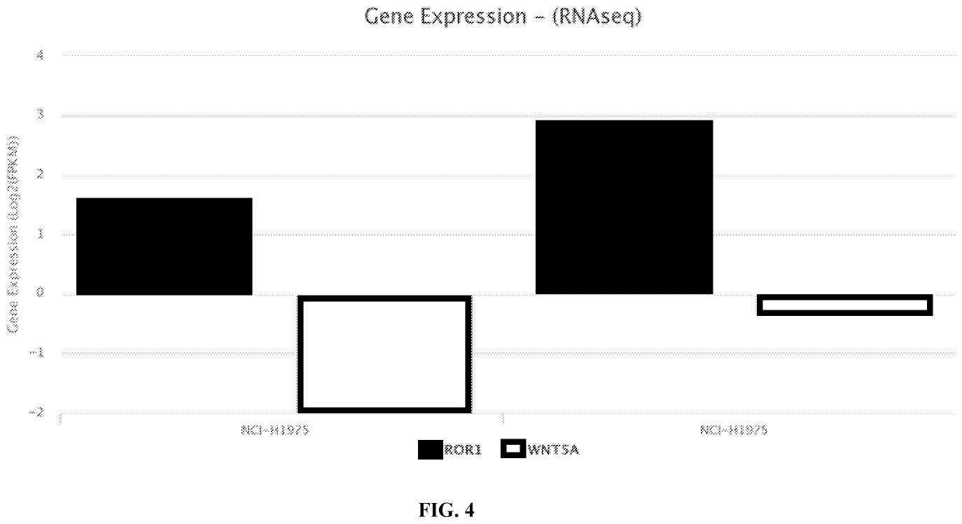

Experiments were performed using NCI-H1975, a NSCLC adenocarcinoma cell line. NCI-H1975 has an L858R mutation that increases EGFR activity. NCI-H1975 also has a T790M mutation in EGFR that confers resistance to first-generation EGFRi (e.g. erlotinib and/or gefitinib) in vitro and in vivo (see FIG. 2 and FIG. 3A-3D). Afatinib, a covalent second-generation EGFRi showed moderate activity against NCI-H1975, and osimertinib showed potent activity against NCI-H1975. NCI-H1975 cells have moderate ROR1 transcript levels, and low (if any) WNT5a gene expression (FIG. 4).

### Cell Culture

NCI-H1975 tumor cells were maintained in in vitro culture in RPMI1640 medium supplemented with 10% fetal bovine serum at 37° C. in an atmosphere of 5% CO₂ in air. The cells in an exponential growth phase were harvested and counted for tumor inoculation.

### Tumor Inoculation

Each mouse was inoculated subcutaneously in the right rear flank region with NCI-H1975 tumor cells (5×10⁶) in 0.1 ml of PBS for tumor development.

### Randomization

For the efficacy study, randomization started when the mean tumor size reached approximately 100-200 mm³. 64 mice were enrolled in the study and randomly allocated to 8 study groups, with 8 mice per group. Tumor volume was used as a numeric parameter to randomize selected animals into specified groups. Randomization was performed based on “Matched distribution” method. The date of randomization was denoted as study day 0, and treatment started at day 0.

### Observation and Data Collection

After tumor cell inoculation, the animals were checked daily for morbidity and mortality. During routine monitoring, the animals were checked for any effects of tumor growth and treatments on behavior such as mobility, food and water consumption, body weight gain/loss (body weights will be measured twice per week after randomization or based on a sponsor's request after randomization), eye/hair matting and any other abnormalities. Mortality and observed clinical signs were recorded for individual animals in detail.

Tumor volumes were measured twice per week in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

The body weights and tumor volumes were measured using Study Director™ software (version 3.1.399.19).

### Study Design and Results

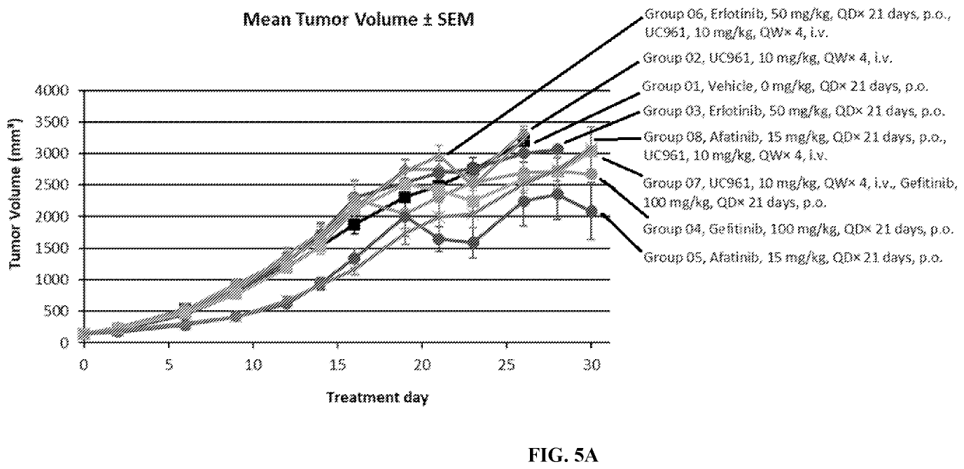

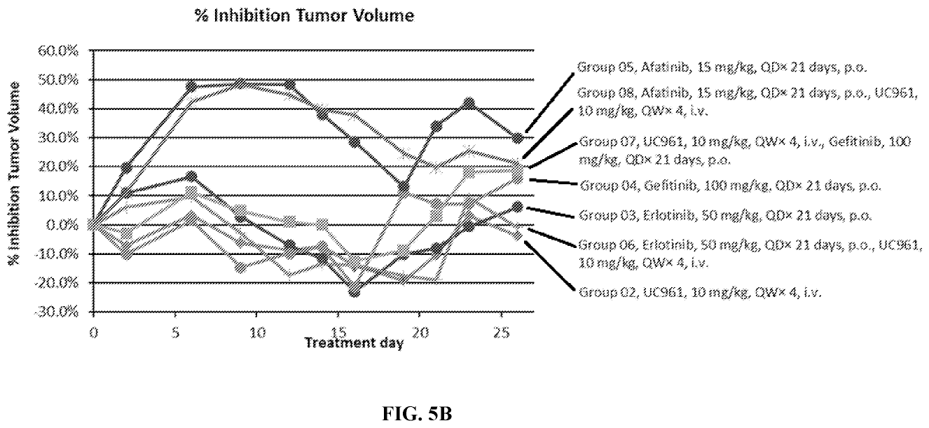

Table 5 shows the study design that was executed for the NCI-H1975 model. In the NCI-H1975 model, the combination of cirmtuzumab (UC-961) and various EGFRi was relatively ineffective except for afatinib which showed a modest anti-tumor effect alone or in combination with cirmtuzumab (FIG. 5A-5B). These results show that although some first or second-generation EGFRi had modest anti-tumor activity, the first and second-generation EGFRi had surprisingly less anti-tumor activity than either osimertinib or osimertinib combined with cirmtuzumab (as shown in a similar model in Example 1), even when the first and second-generation EGFRi were combined with cirmtuzumab. These results highlight the unexpected nature of the beneficial results of combining osimertinib with cirmtuzumab (as shown in Example 1).

TABLE 5
Study design NCI-H1975 Model

| | | | | Dosing | | Dosing |
|---|---|---|---|---|---|---|
| | Animal | | Dose level | Volume | | Frequency & |
| Group | No. | Treatment | (mg/kg) | (μL/g) | ROA | Duration |
| 1 | 8 | Vehicle | — | 10 | p.o. | QD × 21 days |
| 2 | 8 | UC-961 | 10 | 10 | i.v. | QW × 4 |
| 3 | 8 | Erlotinib | 50 | 10 | p.o. | QD × 21 days |
| 4 | 8 | Gefitinib | 100 | 10 | p.o. | QD × 21 days |
| 5 | 8 | Afatinib | 15 | 10 | p.o. | QD × 21 days |
| 6 | 8 | UC-961 | 10 | 10 | i.v. | QW × 4 |
| | | Erlotinib | 50 | 10 | p.o. | QD × 21 days |
| 7 | 8 | UC961 | 10 | 10 | i.v. | QW × 4 |
| | | Gefitinib | 100 | 10 | p.o. | QD × 21 days |
| 8 | 8 | UC-961 | 10 | 10 | i.v. | QW × 4 |
| | | Afatinib | 15 | 10 | p.o. | QD × 21 days |

### Example 3-3^{rd}-Generation EGFR Inhibitor and ROR1 Antagonism Increases Treatment Efficacy in a Mouse Model of NSCLC

The anti-tumor activity of the tyrosine kinase-like orphan receptor 1 (ROR1) antagonist, cirmtuzumab, was tested in combination with the third-generation epidermal growth factor receptor inhibitor (EGFRi), osimertinib, in a patient-derived xenograft (PDX) mouse model of non-small cell lung cancer (NSCLC).

### LU PDX Cell/Lines

Experiments were performed using the LU3075 cell line, which comprises an EGFR exon20-insertion mutation.

### Tumor Inoculation

Fresh tumor tissues from mice bearing established primary human lung cancer PDX model LU3075 were harvested and cut into small pieces (approximately 2-3 mm in diameter). PDX tumor fragments, harvested from donor mice, were inoculated subcutaneously at the upper right dorsal flank into female BALB/c nude mice for tumor development.

### Observation and Data Collection

After tumor inoculation, the animals were checked daily for morbidity and mortality. During routine monitoring, the animals were checked for any effects of tumor growth and treatments on behavior such as mobility, food and water consumption, body weight gain/loss (Body weights would be measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm3 using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using Study Director™ software (version 3.1.399.19).

### Study Design and Results

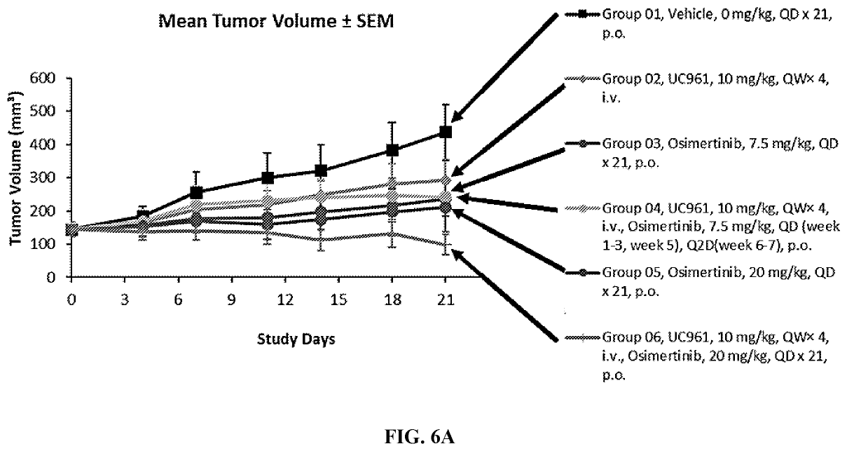

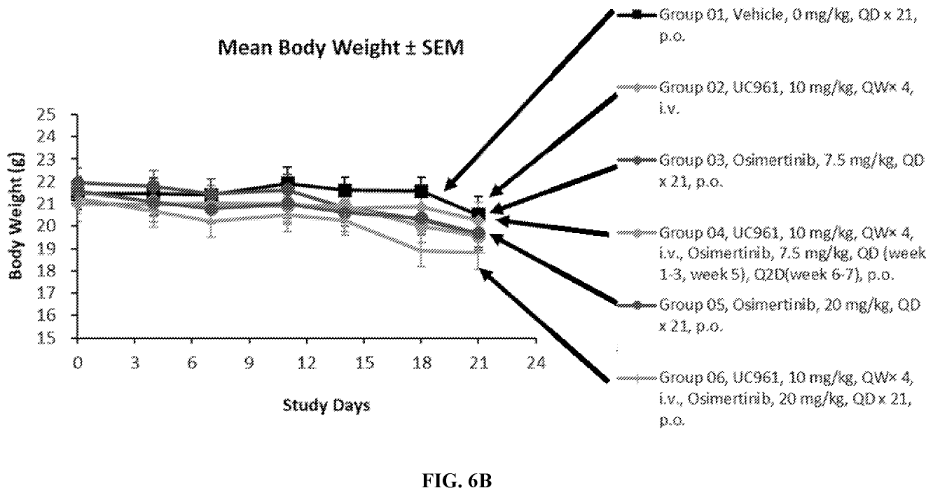

Table 6 shows the study design that was executed for the LU3075 model. In the LU3075 model, the combination of cirmtuzumab (UC-961) and osimertinib showed a synergistic inhibition of tumor growth FIG. 6A and Table 7, and was well tolerated as shown in and FIG. 6B. These data indicate that third-generation EGFRi such as osimertinib show a surprisingly effective synergistic anti-cancer effect when combined with osimertinib.

TABLE 6
Study design for LU3075 Model

| | | | Dose | Dosing | | Original Dosing | Actual Dosing |
|---|---|---|---|---|---|---|---|
| | Animal | | Level | Volume | | Frequency & | Frequency & |
| Group | No. | Treatment | (mg/kg) | (μL/g) | ROA | Duration | Duration# |
| 1 | 8 | Vehicle | — | 10 | p.o. | QD × 21 days | QD (week 1-3, week 5), |
| | | | | | | | Q2D (week 6-7) |
| 2 | 8 | UC961 | 10 | 10 | i.v. | QW × 4 | QW × 7 |
| 3 | 8 | Osimertinib | 7.5 | 10 | p.o. | QD × 21 days | QD (week 1-3, week 5), |
| 4 | 8 | UC961 | 10 | 10 | i.v. | QW × 4 | QW × 7 |
| | | Osimertinib | 7.5 | 10 | p.o. | QD × 21 days | QD (week 1-3, week 5), |
| | | | | | | | Q2D (week 6-7) |
| 5 | 8 | Osimertinib | 20 | 10 | p.o. | QD × 21 days | QD (week 1-3, week 5), |
| | | | | | | | Q2D (week 6-7) |
| 6 | 8 | UC961 | 10 | 10 | i.v. | QW × 4 | QW × 7 |
| | | Osimertinibb | 20 | 10 | p.o. | QD × 21 days | QD (week 1-3, week 5), |
| | | | | | | | Q2D (week 6-7) |

TABLE 7
Efficacy of Osimertinib and UC961 in treating LU3075 Model

| | | On day 21 | | |
|---|---|---|---|---|
| | | Tumor Size | TGI | P |
| Group | Treatment Description | (mm³) | (%) | Value |
| 1 | Vehicle, 0 mg/kg, QD (week 1-3, week 5), Q2D (week 6-7), p.o. | 436.6 ± 83.9 | — | — |
| 2 | UC961, 10 mg/kg, QW × 7, i.v. | 292.3 ± 61.4 | 33.1 | >0.05 |
| 3 | Osimertinib, 7.5 mg/kg, QD (week 1-3, week 5), | 236.4 ± 51.1 | 45.9 | >0.05 |
| | Q2D (week 6-7), p.o. | | | |
| 4 | UC961, 10 mg/kg, QW × 7, i.v., Osimertinib, 7.5 mg/kg, QD | 241.3 ± 46.8 | 44.7 | >0.05 |
| | (week 1-3, week 5), Q2D (week 6-7), p.o. | | | |
| 5 | Osimertinib, 20 mg/kg, QD (week 1-3, week 5), | 210.4 ± 72.5 | 51.8 | >0.05 |
| | Q2D (week 6-7), p.o. | | | |
| 6 | UC961, 10 mg/kg, QW × 7, i.v., Osimertinib, 20 mg/kg, QD | 98.5 ± 31.6 | 77.4 | <0.01 |
| | (week 1-3, week 5), Q2D (week 6-7), p.o. | | | |

TGI = tumor growth inhibition

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Sequence listings provided herein

| SEQ | |
|---|---|
| ID NO: | Sequences Disclosed |
| 1 | GYAFTAYN |
| 2 | FDPYDGGS |
| 3 | GWYYFDY |
| 4 | KSISKY |
| 5 | SGS |
| 6 | QQHDESPY |
| 7 | QVQLQESGPGLVKPSQTLSLTCTVSGYAFTAYNIHWVRQAPGQGLEWMGSF |
| | DPYDGGSSYNQKFKDRLTISKDTSKNQVVLTMTNMDPVDTATYYCARGWY |
| | YFDYWGHGTLVTVSSASTKGPSVFPLAPSSKSTSGGTAALGCLVKDYFPEPVT |
| | VSWNSGALTSGVHTFPAVLQSSGLYSLSSVVTVPSSSLGTQTYICNVNHKPSN |
| | TKVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRW |
| | QQGNVFSCSVMHEALHNHYTQKSLSLSPGK |
| 8 | DIVMTQTPLSLPVTPGEPASISCRASKSISKYLAWYQQKPGQAPRLLIYSGSTL |
| | QSGIPPRFSGSGYGTDFTLTINNIESEDAAYYFCQQHDESPYTFGEGTKVEIKRT |
| | VAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |

What is claimed is:

1. A method of treating cancer in a subject in need thereof, said method comprising administering to said subject a therapeutically effective amount of an epidermal growth factor receptor (EGFR) inhibitor and a tyrosine kinase-like orphan receptor 1 (ROR1) antagonist.

2. The method of claim 1, wherein said EGFR inhibitor is a small molecule.

3. The method of claim 1, wherein said EGFR inhibitor is a third-generation EGFR inhibitor.

4. The method of claim 3, wherein said third-generation EGFR inhibitor is osimertinib, AC0010, lapatinib, mavelertinib, naquotinib, nazartinib, olmutinib, or rociletinib.

5. The method of claim 1, wherein said EGFR inhibitor is osimertinib.

6. The method of claim 1, wherein said ROR1 antagonist is an antibody or a small molecule.

7. The method of claim 6, wherein said antibody comprises a Fab, F(ab′)₂, Fv, or an scFv.

8. The method of claim 1, wherein said ROR1 antagonist is an anti-ROR1 antibody.

9. The method of claim 6, wherein said antibody comprises a humanized heavy chain variable region and a humanized light chain variable region, wherein said humanized heavy chain variable region comprises the sequences set forth in SEQ ID NO:1, SEQ ID NO:2, and SEQ ID NO:3; and wherein said humanized light chain variable region comprises the sequences set forth in SEQ ID NO:4, SEQ ID NO:5, and SEQ ID NO:6.

10. The method of claim 6 wherein said antibody comprises a heavy chain variable region and a light chain variable region, wherein said heavy chain variable region comprises an amino acid sequence at least about 85%, 90%, 95%, 97%, 98%, 99%, or 100 identical to that set forth in SEQ ID NO: 7; and wherein said light chain variable region comprises an amino acid sequence at least about 85%, 90%, 95%, 97%, 98%, 99%, or 100 identical to that set forth in SEQ ID NO: 8.

11. The method of claim 6, wherein said antibody is cirmtuzumab.

12. The method of claim 6, wherein said individual is afflicted with a cancer that comprises a mutated EGFR gene.

13. The method of claim 12, wherein the mutated EGFR gene comprises a mutation resulting in a T790M mutation or an L858R mutation in the EGFR protein or an exon-20 insertion in the EGFR gene.

14. The method of any one of claims 1 to 13, wherein said EGFR inhibitor and said ROR1 antagonist are administered in a combined synergistic amount.

15. The method of any one of claims 1 to 13, wherein said EGFR inhibitor and said ROR1 antagonist are administered substantially simultaneously.

16. The method of any one of claims 1 to 13, wherein said EGFR inhibitor and said ROR1 antagonist are administered separately.

17. The method of any one of claims 1 to 13, wherein said EGFR inhibitor and said ROR1 antagonist are administered in separate compositions.

18. The method of any one of claims 1 to 13, wherein said ROR1 antagonist is administered at a first time point and said EGFR inhibitor is administered at a second time point, wherein said first time point precedes said second time point.

19. The method of any one of claims 1 to 13, wherein said EGFR inhibitor and said ROR1 antagonist are admixed prior to administration.

20. The method of any one of claims 1 to 19, wherein said EGFR inhibitor is administered at an amount from about 20 mg to about 100 mg daily.

21. The method of any one of claims 1 to 19, wherein said EGFR inhibitor is administered at an amount of about 80 mg daily.

22. The method of any one of claims 1 to 19, wherein said EGFR inhibitor is administered at an amount of less than about 80 mg daily.

23. The method of any one of claims 1 to 22, wherein said EGFR inhibitor is administered intravenously.

24. The method of any one of claims 1 to 22, wherein said EGFR inhibitor is administered orally.

25. The method of any one of claims 1 to 24, wherein said EGFR inhibitor is administered daily.

26. The method of any one of claims 1 to 22, wherein said ROR1 antagonist is administered intravenously.

27. The method of any one of claims 1 to 26, wherein said ROR1 antagonist is administered once every two-weeks.

28. The method of any one of claims 1 to 26, wherein said ROR1 antagonist is administered once every three-weeks.

29. The method of any one of claims 1 to 26, wherein said ROR1 antagonist is administered once every four-weeks.

30. The method of any one of claims 1 to 29, wherein said ROR1 antagonist is administered at a dosage from about 200 milligrams to about 800 milligrams.

31. The method of any one of claims 1 to 29, wherein said ROR1 antagonist is administered at a dosage from about 300 milligrams to about 600 milligrams.

32. The method of any one of claims 1 to 29, wherein said ROR1 antagonist is administered at a dosage of about 300 milligrams.

33. The method of any one of claims 1 to 29, wherein said ROR1 antagonist is administered at a dosage of about 600 milligrams.

34. The method of any one of claims 1 to 33, wherein said subject is a mammal.

35. The method of any one of claims 1 to 33, wherein said subject is a human.

36. The method of any one of claims 1 to 35, wherein said cancer is renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, biliary cancer, or adrenal cancer.

37. The method of claim 36, wherein the cancer is colon adenocarcinoma.

38. The method of claim 36, wherein the cancer is cutaneous melanoma.

39. The method of claim 36, wherein the cancer is glioblastoma multiforme.

40. The method of claim 36, wherein the cancer is lung adenocarcinoma.

41. The method of claim 36, wherein the cancer is a non-small cell lung cancer.

42. The method of claim 37, wherein the non-small cell lung cancer comprises a mutation resulting in a T790M mutation or an L858R mutation in the EGFR protein or an exon-20 insertion in the EGFR gene.

43. The method of claim 36, wherein the cancer is a breast cancer.

44. The method of claim 37, wherein the breast cancer is invasive ductal carcinoma.

45. A pharmaceutical composition comprising an EGFR inhibitor of any one of claims 2 to 5, an ROR1 antagonist of any one of claims 8 to 11, and a pharmaceutically acceptable excipient.

46. An epidermal growth factor receptor (EGFR) inhibitor and a tyrosine kinase-like orphan receptor 1 (ROR1) antagonist for use in treating a cancer.

47. The use of claim 46, wherein said EGFR inhibitor is a small molecule.

48. The use of claim 46, wherein said EGFR inhibitor is a third-generation EGFR inhibitor.

49. The use of claim 48, wherein said third-generation EGFR inhibitor is osimertinib, AC0010, lapatinib, mavelertinib, naquotinib, nazartinib, olmutinib, or rociletinib.

50. The use of claim 46, wherein said EGFR inhibitor is osimertinib.

51. The use of claim 46, wherein said ROR1 antagonist is an antibody or a small molecule.

52. The use of claim 51, wherein said antibody comprises a Fab, F(ab′)₂, Fv, or an scFv.

53. The use of claim 46, wherein said ROR1 antagonist is an anti-ROR1 antibody.

54. The use of claim 51, wherein said antibody comprises a humanized heavy chain variable region and a humanized light chain variable region, wherein said humanized heavy chain variable region comprises the sequences set forth in SEQ ID NO:1, SEQ ID NO:2, and SEQ ID NO:3; and wherein said humanized light chain variable region comprises the sequences set forth in SEQ ID NO:4, SEQ ID NO:5, and SEQ ID NO:6.

55. The use of claim 51, wherein said antibody comprises a heavy chain variable region and a light chain variable region, wherein said heavy chain variable region comprises an amino acid sequence at least about 85%, 90%, 95%, 97%, 98%, 99%, or 100 identical to that set forth in SEQ ID NO: 7; and wherein said light chain variable region comprises an amino acid sequence at least about 85%, 90%, 95%, 97%, 98%, 99%, or 100 identical to that set forth in SEQ ID NO: 8.

56. The use of claim 51, wherein said antibody is cirmtuzumab.

57. The use of any one of claims 46 to 51, wherein said individual is afflicted with a cancer that comprises a mutated EGFR gene.

58. The use of claim 57, wherein the mutated EGFR gene comprises a mutation resulting in a T790M mutation or an L858R mutation in the EGFR protein or an exon-20 insertion in the EGFR gene.

59. The use of any one of claims 46 to 58, wherein said cancer is renal cell carcinoma, colon cancer, colorectal cancer, breast cancer, epithelial squamous cell cancer, melanoma, stomach cancer, brain cancer, lung cancer, pancreatic cancer, cervical cancer, ovarian cancer, liver cancer, bladder cancer, prostate cancer, testicular cancer, thyroid cancer, head and neck cancer, uterine cancer, adenocarcinoma, biliary cancer, or adrenal cancer.

60. The use of claim 59, wherein the cancer is colon adenocarcinoma.

61. The use of claim 59, wherein the cancer is cutaneous melanoma.

62. The use of claim 59, wherein the cancer is glioblastoma multiforme.

63. The use of claim 59, wherein the lung cancer is lung adenocarcinoma.

64. The use of claim 59, wherein the cancer is a non-small cell lung cancer.

65. The use of claim 63, wherein the non-small cell lung cancer comprises a mutation.

66. The use of claim 59, wherein the cancer is a breast cancer.

67. The use of claim 66, wherein the breast cancer is invasive ductal carcinoma.

68. The use of any one of claims 46 to 67, wherein said EGFR inhibitor is administered at an amount from about 20 mg to about 100 mg.

69. The use of any one of claims 46 to 67, wherein said EGFR inhibitor is administered at an amount of about 80 mg.

70. The use of any one of claims 46 to 67, wherein said EGFR inhibitor is administered at an amount of less than about 80 mg.

71. The use of any one of claims 46 to 70, wherein said EGFR inhibitor is administered intravenously.

72. The use of any one of claims 46 to 70, wherein said EGFR inhibitor is administered orally.

73. The use of any one of claims 46 to 72, wherein said EGFR inhibitor is administered daily.

74. The use of any one of claims 46 to 73, wherein said ROR1 antagonist is administered intravenously.

75. The use of any one of claims 46 to 74, wherein said ROR1 antagonist is administered once every two-weeks.

76. The use of any one of claims 46 to 74, wherein said ROR1 antagonist is administered once every three-weeks.

77. The use of any one of claims 46 to 74, wherein said ROR1 antagonist is administered once every four-weeks.

78. The use of any one of claims 46 to 77, wherein said ROR1 antagonist is administered at a dosage from about 200 milligrams to about 800 milligrams.

79. The use of any one of claims 46 to 77, wherein said ROR1 antagonist is administered at a dosage from about 300 milligrams to about 600 milligrams.

80. The use of any one of claims 46 to 77, wherein said ROR1 antagonist is administered at a dosage of about 300 milligrams.

81. The use of any one of claims 46 to 77, wherein said ROR1 antagonist is administered at a dosage of about 600 milligrams.
